(19) United States
(12) Patent Application Publication
(10) Pub. No.: US 2025/0072913 A1
(43) Pub. Date: Mar. 6, 2025

(54) ADJUSTABLE SURGICAL GUIDE, VIRTUAL PLANNING, AND SURGICAL NAVIGATION OF SAME

(71) Applicant: Glenoid Solutions, LLC, Tampa, FL (US)

(72) Inventors: Jonathan Levy, Tampa, FL (US); Sergio Gutierrez, Tampa, FL (US)

(73) Assignee: Glenoid Solutions, LLC, Tampa, FL (US)

(21) Appl. No.: 18/582,438

(22) Filed: Feb. 20, 2024

Related U.S. Application Data
(63) Continuation-in-part of application No. 18/411,869, filed on Jan. 12, 2024.
Application No. 18/411,869 is a continuation of application No. 18/240,217, filed on Aug. 30, 2023, now Pat. No. 11,871,942.

(51) Int. Cl. A61B 17/17; A61B 17/00; A61B 17/56

(52) U.S. Cl. CPC A61B 17/17; A61B 17/1778; A61B 2017/00681; A61B 2017/564

(57) ABSTRACT

The technical solutions described herein are systems and methods for adjustable surgical guides, virtual planning, and surgical navigation. The system can process preoperative image data of a glenoid, generate a virtual representation of the glenoid face, and maintain a virtual model of a surgical guide. The system can present the virtual representation and the surgical guide in a user interface, determine version and inclination angles for the surgical guide, and adjust the surgical guide's position or orientation. The system can assess virtual plan data, communicate the data to a surgical system, translate the data into movement commands, and execute the movement commands for drilling. The system can access an intraoperative image of the glenoid face, identify the position of a drilling instrument, generate an image to determine the spatial relationship between the instrument and the virtual representation, and present a dynamic visual indicator to guide drilling.

### CROSS-REFERENCE TO RELATED APPLICATIONS

The present application is a continuation-in part of U.S. application Ser. No. 18/411,869, titled “ADJUSTABLE SURGICAL GUIDE” and filed on Jan. 12, 2024, which is a continuation of U.S. application Ser. No. 18/240,217, titled “ADJUSTABLE SURGICAL GUIDE” and filed on Aug. 30, 2023, the content of each of which is incorporated herein by reference in its entirety.

### FIELD

The present disclosure relates to preoperative surgical planning. More particularly, the present disclosure relates to apparatuses, systems, and methods for generating and utilizing virtual models of surgical guides.

### BACKGROUND

Various types of surgical procedures involve removing tissue (e.g., bone, soft tissue, etc.) from a subject. For example, a surgical procedure may involve drilling into the subject. Surgical guides may be used to facilitate drilling into the subject.

### SUMMARY

According to an aspect of the present disclosure, a surgical guide includes a base and a drill guide coupled to the base, the drill guide including a drill guide body defining a drill bore configured to receive a drill bit and extending through the drill guide, the drill guide being configured to translate relative the base to adjust a drill guide angle defined by the drill bore and the base, the drill guide further being configured to transition between a locked state and an unlocked state, wherein the drill guide angle is fixed in the locked state and the drill guide angle is adjustable in the unlocked state.

According to various embodiments, the base includes a first foot including a first foot end, a second foot including a second foot end, and a third foot including a third foot end, wherein a base plane is defined by the first foot end, the second foot end, and the third foot end, the drill guide angle being defined by the drill bore and the base plane. According to various embodiments, the surgical guide includes a first track coupled to the base and defining a first track opening and a second track coupled to the base and defining a first track opening, and a second track coupled to the base and defining a second track opening, wherein the drill guide is configured to translate relative the base within the first track opening and the second track opening. According to various embodiments, the first track is rotatably coupled to the base and configured to rotate about a first axis. According to various embodiments, the first axis is parallel the base plane. According to various embodiments, the second track is rotatably coupled to the base and configured to rotate about a second axis. According to various embodiments, the second axis is perpendicular the first axis. According to various embodiments, the first axis and the second axis are parallel the base plane. According to various embodiments, drill guide includes a locking nut configured to rotate relative the drill bore, wherein rotation of the locking nut causes the drill guide to transition between the locked stated and the unlocked state. According to various embodiments, the drill guide further includes a clamping member, wherein the rotation of the locking nut in a first direction causes the clamping member to compress against the second track to secure the drill guide in the locked state. According to various embodiments, the drill guide further includes a first projection and a second projection extending from an outer portion of the drill guide body, wherein the rotation of the locking nut in the first direction causes the first projection and the second projection to compress against the first track to secure the drill guide in the locked state. According to various embodiments, the first track opening is a linear opening extending in a direction parallel to the base plane. According to various embodiments, the second track opening is a curved opening defining a convex curvature relative the base plane. According to various embodiments, the drill guide includes an adapter proximate a first end of the drill guide body, the adapter configured to receive a drill guide retainer. According to various embodiments, the adapter includes a plurality of spines surrounding an outer portion of the drill guide body.

According to another aspect of the present disclosure, a surgical system includes a surgical guide including a base and a drill guide coupled to the base, the drill guide including a drill guide body defining a drill bore configured to receive a drill bit and extending through the drill guide, the drill guide being configured to translate relative the base to adjust a drill guide angle defined by the drill bore and the base, the drill guide further being configured to transition between a locked state and an unlocked state, wherein the drill guide angle is fixed in the locked state and the drill guide angle is adjustable in the unlocked state. The surgical system further includes an adjustment device configured to cause a change in the drill guide angle. The adjustment device includes an adjustment base and a drill guide adjuster coupled to the adjustment base the drill guide adjuster including a handle proximate a first drill guide adjuster end of the drill guide adjuster, an adjustment projection proximate a second drill guide adjuster end of the drill guide adjuster, and a drill guide adjuster body positioned between the handle and the adjustment projection, the adjustment projection configured to be received within the drill bore such that movement of the handle causes a change in the drill guide angle. The surgical system further includes an adjustment stand configured to selectively coupled to the surgical guide, wherein the adjustment base is configured to receive at least a portion of the adjustment stand to limit relative movement between the adjustment base and the adjustment stand.

According to various embodiments, the drill guide adjuster further includes a first adjustment track coupled to the adjustment base and defining a first adjustment track opening and a second adjustment track coupled to adjustment body and defining a second adjustment track opening, wherein the drill guide adjuster, wherein movement of the handle causes the drill guide adjuster to translate within at least one of the first adjustment track opening or the second adjustment track opening. According to various embodiments, the first adjustment track is rotatably coupled to the adjustment base and configured to rotate about a first adjustment axis. According to various embodiments, the first adjustment axis is parallel a base plane defined by the base while the drill guide adjuster and the drill guide are coupled to the adjustment stand. According to various embodiments, the second adjustment track is rotatably coupled to the adjustment base and configured to rotate about a second adjustment axis. According to various embodiments, the second adjustment axis is perpendicular the first adjustment axis. According to various embodiments, rotation of the handle is configured to cause the drill guide to transition between the locked state and the unlocked state while the drill guide adjuster is coupled to the drill guide. According to various embodiments, the drill guide includes a locking nut configured to rotate relative the drill bore, wherein the rotation of the locking nut causes the drill guide to transition between the locked stated and the unlocked state. According to various embodiments, the locking nut includes a plurality of locking nut splines and the drill guide adjuster body includes a plurality of drill guide adjuster body splines configured to interface with the plurality of locking nut splines while the drill guide adjuster is coupled to the drill guide such that the rotation of the handle is configured to cause the drill guide to transition between the locked state and the unlocked state. According to various embodiments, the adjustment base defines an adjustment base plane and the drill guide adjuster includes a first adjustment track indicator configured to indicate a first relative angular orientation of the drill guide relative the adjustment base plane. According to various embodiments, the wherein the drill guide adjuster includes a second adjustment track indicator configured to indicate a second relative angular orientation of drill guide relative the adjustment base. According to various embodiments, the adjustment stand includes a first clamping arm and a second clamping arm configured to selectively coupled to the surgical guide to the adjustment stand.

According to another aspect of the present disclosure, a method includes providing an adjustment stand, coupling a surgical guide to the adjustment stand. The surgical guide includes a base, and a drill guide coupled to the base, the drill guide including a drill guide body defining a drill bore and extending through the drill guide, the drill bore and the base defining a drill guide angle. The method further includes providing an adjustment device. The adjustment device includes a drill guide adjuster coupled to the base, the drill guide adjuster including a handle proximate a first drill guide adjuster end of the drill guide adjuster, an adjustment projection proximate a second drill guide adjuster end of the drill guide adjuster, and a drill guide adjuster body positioned between the handle and the adjustment projection. The method further includes inserting the adjustment projection into the drill bore. The method further includes adjusting a position of the handle thereby causing the drill guide to translate relative the base to adjust the drill guide angle. The method further includes locking the drill guide such that the drill guide angle is fixed.

According to various embodiments, the method includes removing the adjustment projection from the drill bore, decoupling the drill guide from the adjustment stand, and providing the drill guide in a desired location proximate a bone. According to various embodiments, the method includes rotating the handle while the adjustment projection is positioned within the drill guide causing the drill guide to transition to a locked state to prevent change in the drill guide angle.

At least one aspect of the technical solutions is directed to a method for virtual surgical planning. The method can include processing, by one or more processors, preoperative image data of a glenoid to identify defined anatomical landmarks. The defined anatomical landmarks can include a glenoid face of the glenoid. The method can include generating, by the one or more processors, a virtual representation of the glenoid face based on the defined anatomical landmarks. The visual representation can include a dynamically adjustable virtual boundary defined on a virtual surface of the glenoid face. The method can include maintaining, by the one or more processors, in a data structure, a virtual model of a surgical guide. The method can include providing, by the one or more processors, for presentation, in a user interface, the virtual representation of the glenoid face and the virtual model of the surgical guide. The virtual model of the surgical guide can be configured to be aligned with the virtual representation of the glenoid face. The virtual model of the surgical guide can include a virtual base plane defined by a number of virtual prongs. The virtual base plane can include at least three virtual prongs. Each virtual prong can be positioned on the virtual surface of the glenoid face within the virtual boundary such that the virtual base plane aligns with a glenoid plane defined by the virtual surface of the glenoid face. In response to aligning the virtual model of the surgical guide with the virtual representation of the glenoid face, the method can include determining, by the one or more processors, a version angle indicating at least one of a backward tilt or a forward tilt of an axis of the surgical guide relative to the glenoid plane and an inclination angle indicating at least one of a downward tilt or an upward tilt of the axis of the surgical guide relative to the glenoid plane. The method can include adjusting, by the one or more processors, a position or orientation of the virtual model of the surgical guide based on at least one of the version angle or the inclination angle.

The method can include presenting, by the one or more processors, for display, a view of the virtual representation of the glenoid face and the virtual model of the surgical guide. The method can include receiving, by the one or more processors, an interaction, via the user interface, to move the position or orientation of the virtual model of the surgical guide. The interaction can be received via an interactive element, a button, a voice command, a joystick, a gesture recognition system, or tactile/haptic feedback. The method can include modifying, by the one or more processors, in response to receiving the interaction, the view of the virtual representation of the glenoid face and the virtual model of the surgical guide, where the view can be at least one of a coronal view, a sagittal view, or a transverse view.

The method can include restricting, by the one or more processors, positioning of the virtual model of the surgical guide within the virtual boundary. The method can include providing, by the one or more processors, feedback in response to determining that the virtual model of the surgical guide extends outside the virtual boundary. The feedback can include at least one of an error message or a visual indication.

The method can include receiving, by the one or more processors, an input, via the user interface, to adjust the position or orientation of the virtual model of the surgical guide. In response to receiving the input, the method can include generating, by the one or more processors, an output identifying the version angle and the inclination angle.

The method can include automatically positioning, by the one or more processors, the virtual prongs of the virtual model of the surgical guide at a first set of determined prong locations on the virtual surface of the glenoid face. The method can include automatically repositioning, by the one or more processors, the virtual prongs of the virtual model of the surgical guide to a second set of determined prong locations on the virtual surface of the glenoid face in response to adjusting the position or orientation of the virtual model of the surgical guide. The surgical guide can include a base with at least three feet defining a base plane. The surgical guide can include a drill guide coupled to the base. The drill guide can include a drill guide body that defines a drill bore configured to receive a drill bit and extend through the drill guide. The drill guide can translate relative to the base to adjust a drill guide angle, which can be defined by the drill bore and the base. Additionally, the drill guide can transition between a locked state and an unlocked state, where the drill guide angle is fixed in the locked state and adjustable in the unlocked state. The surgical guide can include a first track coupled to the base, defining a first track opening, and a second track coupled to the base, defining a second track opening. The drill guide can translate relative to the base within the first and second track openings.

The method can include indicating, by the one or more processors, varying anatomical densities on the virtual representation of the glenoid face with color-coded regions. The varying anatomical densities can be determined from the preoperative image data of the glenoid.

The method can include determining, by the one or more processors, the version angle and the inclination angle based on biomechanical data indicating a patient-specific glenoid anatomy.

The method can include determining, by the one or more processors, a spatial relationship between the virtual model of the surgical guide and the virtual representation of the glenoid face. The spatial relationship can include one or more spatial parameters indicating at least one of a distance between the virtual base plane of the surgical guide and the glenoid plane, orientation angles, intersection points, or areas of overlap.

At least one aspect of the technical solutions is directed to a method for executing a virtual surgical plan. The method can include assessing, by one or more processors, virtual plan data. The virtual plan data can include a drilling location based on defined anatomical landmarks along a pathway to provide an opening to implant a surgical implant in a glenoid, and a version angle and an inclination angle of a virtual model of a surgical guide positioned according to the drilling location relative to a virtual representation of a glenoid face. The method can include communicating, by the one or more processors, the virtual plan data to a surgical system. The surgical system can be configured to translate the virtual plan data into defined movement commands. The defined movement commands can indicate a placement of the surgical guide based on the virtual plan data. The surgical system can be configured to execute the defined movement commands to cause the surgical system to drill at the drilling location.

The method can include validating, by the one or more processors, the virtual plan data according to preoperative data. To translate the virtual plan data into defined movement commands, the surgical system can be further configured to identify a position or orientation of the virtual model of the surgical guide relative to the virtual representation of the glenoid face. The virtual model of the surgical guide can include a number of virtual prongs positioned on a virtual surface of the glenoid face. The method can include causing, by the one or more processors, the surgical system to restrict a drilling instrument within a virtual boundary of the glenoid face.

At least one aspect of the technical solutions is directed to a method for navigating according to a virtual surgical plan. The method can include accessing, by one or more processors, an intraoperative image of a glenoid face. The method can include identifying, by the one or more processors, a position of a drilling instrument coupled to a surgical guide within the intraoperative image. The method can include generating, by the one or more processors, an image by overlapping a virtual representation of the glenoid face onto the intraoperative image. The method can include determining, by the one or more processors, using the image, a spatial relationship between the position of the drilling instrument and the virtual representation of the glenoid face based on a version angle and an inclination angle of the surgical guide. The spatial relationship can correspond to an offset between the position of the drilling instrument and the drilling location on the glenoid face. The method can include presenting, by the one or more processors, a dynamic visual indicator on the image indicating a drilling location to place the drilling instrument. The position of the drilling instrument coupled to the surgical guide can be identified based on defined marker patterns of the surgical guide within the intraoperative image. The defined marker patterns of the surgical guide can include geometric shapes, alphanumeric characters, or color-coded markers.

The method can include identifying, by the one or more processors, anatomical landmarks in the virtual representation of the glenoid face and the intraoperative image of the glenoid face. The method can include aligning, by the one or more processors, the anatomical landmarks in the virtual representation of the glenoid face with corresponding landmarks in the intraoperative image of the glenoid face.

The method can include presenting, by the one or more processors, the dynamic visual indicator by projecting a light pattern directly onto the glenoid face, the light pattern visually indicating the drilling location, overlaying the drilling location onto a surgeon's view via an augmented reality, virtual reality, or mixed reality headset, or presenting the drilling location in relation to the virtual representation of the glenoid face on a monitor or a screen. The method can include accessing, by the one or more processors, a time-stamped sequence of the intraoperative images of the glenoid face in response to identifying movement of the drilling instrument.

At least one aspect of the technical solutions is directed to a system for virtual surgical planning. The system can include one or more processors coupled with memory. The one or more processors can be configured to process preoperative image data of a glenoid to identify defined anatomical landmarks. The defined anatomical landmarks can include a glenoid face of the glenoid. The one or more processors can be configured to generate a virtual representation of the glenoid face based on the defined anatomical landmarks. The visual representation can include a dynamically adjustable virtual boundary defined on a virtual surface of the glenoid face. The one or more processors can be configured to maintain, in a data structure, a virtual model of a surgical guide. The one or more processors can be configured to provide, for presentation, in a user interface, the virtual representation of the glenoid face and the virtual model of the surgical guide. The virtual model of the surgical guide can be configured to be aligned with the virtual representation of the glenoid face. The virtual model of the surgical guide can include a virtual base plane defined by a number of virtual prongs. The virtual base plane can include at least three virtual prongs. Each virtual prong can be positioned on the virtual surface of the glenoid face within the virtual boundary such that the virtual base plane aligns with a glenoid plane defined by the virtual surface of the glenoid face. In response to aligning the virtual model of the surgical guide with the virtual representation of the glenoid face, the one or more processors can be configured to determine a version angle indicating at least one of a backward tilt or a forward tilt of an axis of the surgical guide relative to the glenoid plane and an inclination angle indicating at least one of a downward tilt or an upward tilt of the axis of the surgical guide relative to the glenoid plane. The one or more processors can be configured to adjust a position or orientation of the virtual model of the surgical guide based on at least one of the version angle or the inclination angle.

The one or more processors can be configured to present, for display, a view of the virtual representation of the glenoid face and the virtual model of the surgical guide. The one or more processors can be configured to receive an interaction, via the user interface, to move the position or orientation of the virtual model of the surgical guide. The interaction can be received via an interactive element, a button, a voice command, a joystick, a gesture recognition system, or tactile/haptic feedback. The one or more processors can be configured to modify, in response to receiving the interaction, the view of the virtual representation of the glenoid face and the virtual model of the surgical guide, where the view can be at least one of a coronal view, a sagittal view, or a transverse view.

The one or more processors can be configured to restrict the positioning of the virtual model of the surgical guide within the virtual boundary. The one or more processors can be configured to provide feedback in response to determining that the virtual model of the surgical guide extends outside the virtual boundary. The feedback can include at least one of an error message or a visual indication.

The one or more processors can be configured to receive, an input, via the user interface, to adjust the position or orientation of the virtual model of the surgical guide. In response to receiving the input, the one or more processors can be configured to generate an output identifying the version angle and the inclination angle.

The one or more processors can be configured to automatically position the virtual prongs of the virtual model of the surgical guide at a first set of determined prong locations on the virtual surface of the glenoid face. The one or more processors can be configured to automatically reposition the virtual prongs of the virtual model of the surgical guide to a second set of determined prong locations on the virtual surface of the glenoid face in response to adjusting the position or orientation of the virtual model of the surgical guide. The surgical guide can include a base with at least three feet defining a base plane. The surgical guide can include a drill guide coupled to the base. The drill guide can include a drill guide body that defines a drill bore configured to receive a drill bit and extend through the drill guide. The drill guide can translate relative to the base to adjust a drill guide angle, which can be defined by the drill bore and the base. Additionally, the drill guide can transition between a locked state and an unlocked state, where the drill guide angle is fixed in the locked state and adjustable in the unlocked state. The surgical guide can include a first track coupled to the base, defining a first track opening, and a second track coupled to the base, defining a second track opening. The drill guide can translate relative to the base within the first and second track openings.

The one or more processors can be configured to indicate varying anatomical densities on the virtual representation of the glenoid face with color-coded regions. The varying anatomical densities can be determined from the preoperative image data of the glenoid. The one or more processors can be configured to determine the version angle and the inclination angle based on biomechanical data indicating a patient-specific glenoid anatomy.

The one or more processors can be configured to determine a spatial relationship between the virtual model of the surgical guide and the virtual representation of the glenoid face. The spatial relationship can include one or more spatial parameters indicating at least one of a distance between the virtual base plane of the surgical guide and the glenoid plane, orientation angles, intersection points, or areas of overlap.

At least one aspect of the technical solutions is directed to a system for executing a virtual surgical plan. The system can include one or more processors coupled with memory. The one or more processors can be configured to assess virtual plan data. The virtual plan data can include a drilling location based on defined anatomical landmarks along a pathway to provide an opening to implant a surgical implant in a glenoid, and a version angle and an inclination angle of a virtual model of a surgical guide positioned according to the drilling location relative to a virtual representation of a glenoid face. The one or more processors can be configured to communicate the virtual plan data to a surgical system. The surgical system can be configured to translate the virtual plan data into defined movement commands. The defined movement commands can indicate a placement of the surgical guide based on the virtual plan data. The surgical system can be configured to execute the defined movement commands to cause the surgical system to drill at the drilling location.

The one or more processors can be configured to validate the virtual plan data according to preoperative data. To translate the virtual plan data into defined movement commands, the surgical system can be further configured to identify a position or orientation of the virtual model of the surgical guide relative to the virtual representation of the glenoid face. The virtual model of the surgical guide can include a number of virtual prongs positioned on a virtual surface of the glenoid face. The one or more processors can be configured to cause the surgical system to restrict a drilling instrument within a virtual boundary of the glenoid face.

At least one aspect of the technical solutions is directed to a system for navigating according to a virtual surgical plan. The system can include one or more processors coupled with memory. The one or more processors can be configured to access an intraoperative image of a glenoid face. The one or more processors can be configured to identify a position of a drilling instrument coupled to a surgical guide within the intraoperative image. The one or more processors can be configured to generate an image by overlapping a virtual representation of the glenoid face onto the intraoperative image. The one or more processors can be configured to determine, using the image, a spatial relationship between the position of the drilling instrument and the virtual representation of the glenoid face based on a version angle and an inclination angle of the surgical guide. The spatial relationship can correspond to an offset between the position of the drilling instrument and the drilling location on the glenoid face. The one or more processors can be configured to present a dynamic visual indicator on the image indicating a drilling location to place the drilling instrument. The position of the drilling instrument coupled to the surgical guide can be identified based on defined marker patterns of the surgical guide within the intraoperative image. The defined marker patterns of the surgical guide can include geometric shapes, alphanumeric characters, or color-coded markers.

The one or more processors can be configured to identify anatomical landmarks in the virtual representation of the glenoid face and the intraoperative image of the glenoid face. The one or more processors can be configured to align the anatomical landmarks in the virtual representation of the glenoid face with corresponding landmarks in the intraoperative image of the glenoid face.

The one or more processors can be configured to present the dynamic visual indicator by projecting a light pattern directly onto the glenoid face, the light pattern visually indicating the drilling location, overlaying the drilling location onto a surgeon's view via an augmented reality, virtual reality, or mixed reality headset, or presenting the drilling location in relation to the virtual representation of the glenoid face on a monitor or a screen. The one or more processors can be configured to access a time-stamped sequence of the intraoperative images of the glenoid face in response to identifying the movement of the drilling instrument.

An aspect of the technical solutions is directed to a non-transitory computer readable medium storing instructions. The instructions can be such that, when executed by one or more processors, cause the one or more processors to process preoperative image data of a glenoid to identify defined anatomical landmarks. The defined anatomical landmarks can include a glenoid face of the glenoid. The instructions can be such that, when executed by one or more processors, cause the one or more processors to generate a virtual representation of the glenoid face based on the defined anatomical landmarks. The visual representation can include a dynamically adjustable virtual boundary defined on a virtual surface of the glenoid face. The instructions can be such that, when executed by one or more processors, cause the one or more processors to maintain, in a data structure, a virtual model of a surgical guide. The instructions can be such that, when executed by one or more processors, cause the one or more processors to provide, for presentation, in a user interface, the virtual representation of the glenoid face and the virtual model of the surgical guide. The virtual model of the surgical guide can be configured to be aligned with the virtual representation of the glenoid face. The virtual model of the surgical guide can include a virtual base plane defined by a number of virtual prongs. The virtual base plane can include at least three virtual prongs. Each virtual prong can be positioned on the virtual surface of the glenoid face within the virtual boundary such that the virtual base plane aligns with a glenoid plane defined by the virtual surface of the glenoid face. In response to aligning the virtual model of the surgical guide with the virtual representation of the glenoid face, the instructions can be such that, when executed by one or more processors, cause the one or more processors to determine a version angle indicating at least one of a backward tilt or a forward tilt of an axis of the surgical guide relative to the glenoid plane and an inclination angle indicating at least one of a downward tilt or an upward tilt of the axis of the surgical guide relative to the glenoid plane. The instructions can be such that, when executed by one or more processors, cause the one or more processors to adjust a position or orientation of the virtual model of the surgical guide based on at least one of the version angle or the inclination angle.

The instructions can be such that, when executed by one or more processors, cause the one or more processors to present, for display, a view of the virtual representation of the glenoid face and the virtual model of the surgical guide. The instructions can be such that, when executed by one or more processors, cause the one or more processors to receive an interaction, via the user interface, to move the position or orientation of the virtual model of the surgical guide. The interaction can be received via an interactive element, a button, a voice command, a joystick, a gesture recognition system, or tactile/haptic feedback. The instructions can be such that, when executed by one or more processors, cause the one or more processors to modify, in response to receiving the interaction, the view of the virtual representation of the glenoid face and the virtual model of the surgical guide, where the view can be at least one of a coronal view, a sagittal view, or a transverse view.

The instructions can be such that, when executed by one or more processors, cause the one or more processors to restrict the positioning of the virtual model of the surgical guide within the virtual boundary. The instructions can be such that, when executed by one or more processors, cause the one or more processors to provide feedback in response to determining that the virtual model of the surgical guide extends outside the virtual boundary. The feedback can include at least one of an error message or a visual indication.

The instructions can be such that, when executed by one or more processors, cause the one or more processors to receive, an input, via the user interface, to adjust the position or orientation of the virtual model of the surgical guide. In response to receiving the input, the instructions can be such that, when executed by one or more processors, cause the one or more processors to generate an output identifying the version angle and the inclination angle.

The instructions can be such that, when executed by one or more processors, cause the one or more processors to automatically position the virtual prongs of the virtual model of the surgical guide at a first set of determined prong locations on the virtual surface of the glenoid face. The instructions can be such that, when executed by one or more processors, cause the one or more processors to automatically reposition the virtual prongs of the virtual model of the surgical guide to a second set of determined prong locations on the virtual surface of the glenoid face in response to adjusting the position or orientation of the virtual model of the surgical guide.

The instructions can be such that, when executed by one or more processors, cause the one or more processors to indicate varying anatomical densities on the virtual representation of the glenoid face with color-coded regions. The varying anatomical densities can be determined from the preoperative image data of the glenoid. The instructions can be such that, when executed by one or more processors, cause the one or more processors to determine the version angle and the inclination angle based on biomechanical data indicating a patient-specific glenoid anatomy.

The instructions can be such that, when executed by one or more processors, cause the one or more processors to determine a spatial relationship between the virtual model of the surgical guide and the virtual representation of the glenoid face. The spatial relationship can include one or more spatial parameters indicating at least one of a distance between the virtual base plane of the surgical guide and the glenoid plane, orientation angles, intersection points, or areas of overlap.

An aspect of the technical solutions is directed to a non-transitory computer readable medium storing instructions. The instructions can be such that, when executed by one or more processors, cause the one or more processors to assess virtual plan data. The virtual plan data can include a drilling location based on defined anatomical landmarks along a pathway to provide an opening to implant a surgical implant in a glenoid, and a version angle and an inclination angle of a virtual model of a surgical guide positioned according to the drilling location relative to a virtual representation of a glenoid face. The instructions can be such that, when executed by one or more processors, cause the one or more processors to communicate the virtual plan data to a surgical system. The surgical system can be configured to translate the virtual plan data into defined movement commands. The defined movement commands can indicate a placement of the surgical guide based on the virtual plan data. The surgical system can be configured to execute the defined movement commands to cause the surgical system to drill at the drilling location.

The instructions can be such that, when executed by one or more processors, cause the one or more processors to validate the virtual plan data according to preoperative data. To translate the virtual plan data into defined movement commands, the surgical system can be further configured to identify a position or orientation of the virtual model of the surgical guide relative to the virtual representation of the glenoid face. The virtual model of the surgical guide can include a number of virtual prongs positioned on a virtual surface of the glenoid face. The instructions can be such that, when executed by one or more processors, cause the one or more processors to cause the surgical system to restrict a drilling instrument within a virtual boundary of the glenoid face.

An aspect of the technical solutions is directed to a non-transitory computer readable medium storing instructions. The instructions can be such that, when executed by one or more processors, cause the one or more processors to access an intraoperative image of a glenoid face. The instructions can be such that, when executed by one or more processors, cause the one or more processors to identify a position of a drilling instrument coupled to a surgical guide within the intraoperative image. The instructions can be such that, when executed by one or more processors, cause the one or more processors to generate an image by overlapping a virtual representation of the glenoid face onto the intraoperative image. The instructions can be such that, when executed by one or more processors, cause the one or more processors to determine, using the image, a spatial relationship between the position of the drilling instrument and the virtual representation of the glenoid face based on a version angle and an inclination angle of the surgical guide. The spatial relationship can correspond to an offset between the position of the drilling instrument and the drilling location on the glenoid face. The instructions can be such that, when executed by one or more processors, cause the one or more processors to present a dynamic visual indicator on the image indicating a drilling location to place the drilling instrument. The position of the drilling instrument coupled to the surgical guide can be identified based on defined marker patterns of the surgical guide within the intraoperative image. The defined marker patterns of the surgical guide can include geometric shapes, alphanumeric characters, or color-coded markers.

The instructions can be such that, when executed by one or more processors, cause the one or more processors to identify anatomical landmarks in the virtual representation of the glenoid face and the intraoperative image of the glenoid face. The instructions can be such that, when executed by one or more processors, cause the one or more processors to align the anatomical landmarks in the virtual representation of the glenoid face with corresponding landmarks in the intraoperative image of the glenoid face.

The instructions can be such that, when executed by one or more processors, cause the one or more processors to present the dynamic visual indicator by projecting a light pattern directly onto the glenoid face, the light pattern visually indicating the drilling location, overlaying the drilling location onto a surgeon's view via an augmented reality, virtual reality, or mixed reality headset, or presenting the drilling location in relation to the virtual representation of the glenoid face on a monitor or a screen. The instructions can be such that, when executed by one or more processors, cause the one or more processors to access a time-stamped sequence of the intraoperative images of the glenoid face in response to identifying the movement of the drilling instrument.

This summary is illustrative only and should not be regarded as limiting.

### BRIEF DESCRIPTION OF THE DRAWINGS

FIG. 1 is a perspective view of a surgical guide, according to an example embodiment;

FIG. 2 is another perspective view of the surgical guide of FIG. 1;

FIG. 3 is another perspective view of the surgical guide of FIG. 1;

FIG. 4 is another perspective view of the surgical guide of FIG. 1;

FIG. 5 is a perspective view of a drill guide body, according to an example embodiment;

FIG. 6 is an exploded view of the drill guide body of FIG. 5;

FIG. 7 is a perspective view of a surgical system, according to an example embodiment;

FIG. 8 is an exploded view of the surgical system of FIG. 7;

FIG. 9 is a perspective view of a drill guide adjuster, according to an example embodiment;

FIG. 10 is another perspective view of the drill guide adjuster of FIG. 9;

FIG. 11 is a perspective view of an adjustment stand, according to an example embodiment;

FIG. 12 is a perspective view of the surgical guide of FIG. 1 positioned on top of the adjustment stand of FIG. 11;

FIG. 13 is a perspective view of the surgical guide of FIG. 1 coupled to the adjustment stand of FIG. 11;

FIG. 14 is a perspective view of the drill guide of FIG. 1 positioned proximate a tissue, according to an example embodiment;

FIG. 15 is a perspective view of a drill guide retainer coupled to the drill guide of FIG. 1, according to an example embodiment;

FIG. 16 is a perspective view of another drill guide retainer coupled to the drill guide of FIG. 1, according to an example embodiment;

FIG. 17 is a cross sectional view of the drill guide retainer of FIG. 16 coupled to the drill guide of FIG. 1;

FIG. 18 is a block diagram illustrating a method of using the surgical guide of FIG. 1, according to an example embodiment;

FIG. 19 illustrates a block diagram of an example system for providing virtual surgical planning, in accordance with one or more implementations;

FIGS. 20A-20D illustrate a user interface displaying several perspective views of a virtual model of a surgical guide, in accordance with one or more implementations;

FIG. 21 illustrates a block diagram of an example system for executing and navigating a virtual surgical plan, in accordance with one or more implementations;

FIG. 22 illustrates an example flow diagram of a method for providing virtual surgical planning, in accordance with one or more implementations;

FIG. 23 illustrates an example flow diagram of a method for executing a virtual surgical plan, in accordance with one or more implementations;

FIG. 24 illustrates an example flow diagram of a method for navigating a virtual surgical plan, in accordance with one or more implementations; and

FIG. 25 illustrates a block diagram of a server system and a client computer system in accordance with an illustrative implementation.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

The use of “e.g.” “etc.,” “for instance,” “in example,” and “or” and grammatically related terms indicates non-exclusive alternatives without limitation, unless otherwise noted. The use of “optionally” and grammatically related terms means that the subsequently described element, event, feature, or circumstance may or may not be present/occur, and that the description includes instances where said element, event, feature, or circumstance occurs and instances where it does not. The use of “attached” and “coupled” and grammatically related terms refers to the fixed, releasable, or integrated association of two or more elements and/or devices with or without one or more other elements in between. Thus, the term “attached” or “coupled” and grammatically related terms include releasably attaching or fixedly attaching two or more elements and/or devices in the presence or absence of one or more other elements in between.

Surgical procedures can involve removing tissue (e.g., bone, soft tissue, etc.) from a subject. For example, a surgical procedure may involve drilling into the tissue the subject. Drilling into the tissue of the subject may involve drilling through a surgical guide, which may guide a drill bit into a desired location at a desired orientation. While surgical guides can improve accuracy of the drilling, the surgical guide may need to be specific to the subject of the surgical procedure. For example, due to various factors (e.g., the anatomy of the subject, the severity or type of injury being addressed, the condition of the tissue being drilled into, etc.), the surgical guide may need to be specific for the surgical procedure and the subject for optimal results. However, creating a custom surgical guide for each procedure may be a timely and expensive process.

Referring to the figures generally, a surgical guide is shown, according to an example embodiment. The surgical guide is configured to be quickly and easily adjusted (e.g., using an adjustment stand and a drill guide adjuster) such that tissue may be removed in a desired location and at a desired orientation (e.g., angle). For example, a desired or optimal drill angle (e.g., a desired orientation of a hole being drilled into a bone of the subject relative a surface of the bone) may be determined prior to the procedure and a user of the surgical guide may set a drill guide angle (e.g., an angle defined by a drill bore of the drill guide and a base plane defined by a base of the surgical guide) based on the optimal drill angle. The surgical guide may then be positioned in a desired location proximate the tissue of the subject and a hole may be drilled through the drill bore of the surgical guide.

According to various embodiments, the drill guide is reusable. For example, the drill guide may be formed of a material that will not deteriorate during a sanitation process (e.g., within an autoclave). Thus, the drill guide may be customizable for a desired procedure and reusable to reduce cost. Additionally, or alternatively, one or more of the other components of the surgical systems described herein may be reusable (e.g., the drill guide adjustor and/or the adjustment stand).

According to various embodiments described herein, a surgical guide includes a base defining a base plane. The base may be configured to engage tissue of a subject of a surgical procedure. The surgical guide further includes a first track coupled to the base and defining a first track opening and a second track coupled to the base and defining a second track opening. A drill guide may be coupled to at least one of the first track or the second track. For example, the drill guide may extend through the first track opening and the second track opening such that movement of the drill guide is restricted by the first track and/or the second track. The drill guide may include a drill guide body defining a drill bore that extends along a drill bore axis through the drill guide body. The drill bore may be configured to receive a drill bit such that the drill bit can be inserted through the drill guide to remove tissue from the subject. The drill guide may be configured to translate relative the base within the first track opening and the second track opening to adjust a drill guide angle defined by the drill bore and the base plane. For example, a user of the drill guide may change the drill guide angle based on a determined optimal drill angle prior to drilling through the drill guide.

According to various embodiments, the base includes a first foot including a first foot end, a second foot including a second foot end, and a third foot including a third foot end, wherein the base plane is defined by the first foot end, the second foot end, and the third foot end. Each of the first foot end, the second foot end, and the third foot end may come to a pointed end such that the base plane extends through each of the pointed ends.

According to various embodiments, the first track is rotatably coupled to the base and configured to rotate about a first axis. The first axis may be parallel the base plane. The second track is rotatably coupled to the base and configured to rotate about a second axis. The second axis may be perpendicular the first axis. Further, the first axis and the second axis may be parallel the base plane.

### A. Surgical Guide

According to various embodiments, the drill guide is configured to transition between a locked state and an unlocked state. The drill guide angle may be fixed (e.g., locked, restricted, etc.) in the locked state and the drill guide angle may be adjustable in the unlocked state. The drill guide may include a locking nut configured to rotate relative the drill bore, wherein rotation of the locking nut causes the drill guide to transition between the locked stated and the unlocked state. The drill guide further may include a clamping member configured to secure the drill guide in the locked state. For example, rotation of the locking nut in a first direction may cause the clamping member to compress against the second track to secure the drill guide in the locked state. The drill guide further may further include a first projection and a second projection extending from an outer portion of the drill guide body. The rotation of the locking nut in the first direction may causes the first projection and the second projection to compress against the first track (e.g., in an opposite direction as the clamping member) to further secure the drill guide in the locked state.

According to various embodiments, the first track opening is a linear opening extending in a direction parallel to the base plane. Further, the second track opening may be a curved opening defining a convex curvature relative the base plane.

According to various embodiments, the drill guide includes an adapter proximate a first end of the drill guide body. The adapter may be configured to receive a drill guide retainer or a drill guide reducer. The adapter may include a plurality of spines surrounding an outer portion of the drill guide body.

According to various embodiments, a surgical system includes surgical guide. The surgical guide may include a base defining a base plane, a first track coupled to the base and defining a first track opening, a second track coupled to the base and defining a second track opening, and a drill guide coupled to at least one of the first track or the second track and extending through the first track opening and the second track opening. The drill guide may include a drill guide body defining a drill bore configured to receive a drill bit. The drill guide may be configured to translate relative the base within the first track opening and the second track opening to adjust a drill guide angle defined by the drill bore and the base plane.

According to various embodiments, the surgical system includes an adjustment device configured to cause a change in the drill guide angle. The adjustment device may include one or more angular indicators configured to provide visual feedback to a user of the adjustment device, thereby allowing the user to precisely set the desired drill guide angle. The adjustment device may include an adjustment base, a first adjustment track coupled to the adjustment base and defining a first adjustment track opening, a second adjustment track coupled to adjustment body and defining a second adjustment track opening, and a drill guide adjuster coupled to at least one of the first adjustment track or the second adjustment track. The drill guide adjuster may extend through the first adjustment track opening and the second adjustment track opening. The drill guide adjuster may include a handle proximate a first drill guide adjuster end of the drill guide adjuster. The drill guide adjuster may include an adjustment projection proximate a second drill guide adjuster end of the drill guide adjuster, and a drill guide adjuster body positioned between the handle and the adjustment projection. The adjustment projection may be configured to be received within the drill bore such that movement of the handle causes a change in the drill guide angle, thereby allowing the user of the adjustment device to adjust the drill guide angle.

According to various embodiments, the surgical system includes an adjustment stand. The adjustment stand may be configured to selectively coupled to the surgical guide. Further, the adjustment base may be configured to receive at least a portion of the adjustment stand to limit relative movement between the adjustment base and the adjustment stand. For example, the adjustment device may be placed on top of the adjustment stand and a portion of the adjustment stand base may be contained within the adjuster base to prevent relative movement therebetween.

According to various embodiments, the first adjustment track is rotatably coupled to the adjustment base and configured to rotate about a first adjustment axis. The first adjustment axis may be parallel to the base plane while the drill guide adjuster and the drill guide are coupled to the adjustment stand. The second adjustment track may be rotatably coupled to the adjustment base and configured to rotate about a second adjustment axis. The second adjustment axis may be perpendicular to the first adjustment axis.

According to various embodiments, the drill guide is configured to transition between a locked state and an unlocked state, wherein the drill guide angle is fixed in the locked state and the drill guide angle is adjustable in the unlocked state. When the adjustment projection is positioned within the drill bore, rotation of the handle may to cause the drill guide to transition between the locked state and the unlocked state. For example, the drill guide may include a locking nut configured to rotate relative the drill bore, wherein the rotation of the locking nut causes the drill guide to transition between the locked stated and the unlocked state. The locking nut may include a plurality of locking nut splines and the drill guide adjuster body includes a plurality of drill guide adjuster body splines configured to interface with the plurality of locking nut splines while the drill guide adjuster is coupled to the drill guide such that the rotation of the handle is configured to cause the drill guide to transition between the locked state and the unlocked state.

According to various embodiments, the first adjustment track opening is a linear opening, and the second track opening is a curved opening defining a convex curvature. The adjustment base may define an adjustment base plane and the drill guide adjuster includes a first adjustment track indicator configured to indicate a first relative angular orientation of the drill guide (e.g., within a first adjustment plane) relative the adjustment base plane. The drill guide adjuster may include a second adjustment track indicator configured to indicate a second relative angular orientation of the drill guide (e.g., within a second adjustment plane) relative the adjustment base. The adjustment stand may include a first clamping arm and a second clamping arm configured to selectively coupled to the surgical guide to the adjustment stand.

According to various embodiments, a method of using a surgical guide includes providing an adjustment stand and coupling a surgical guide to the adjustment stand. The surgical guide may include a base defining a base plane, a first track coupled to the base and defining a first track opening, a second track coupled to the base and defining a second track opening, and a drill guide coupled to at least one of the first track or the second track and extending through the first track opening and the second track opening. The drill guide may include a drill guide body defining a drill bore and extending through the drill guide, the drill bore and the base plane defining a drill guide angle. The method may further include providing an adjustment device. The adjustment device may include an adjustment base, a first adjustment track coupled to the adjustment base and defining a first adjustment track opening, a second adjustment track coupled to adjustment body and defining a second adjustment track opening, and a drill guide adjuster coupled to at least one of the first adjustment track or the second adjustment track and extending through the first adjustment track opening and the second adjustment track opening. The drill guide adjuster may include a handle proximate a first drill guide adjuster end of the drill guide adjuster, an adjustment projection proximate a second drill guide adjuster end of the drill guide adjuster, and a drill guide adjuster body positioned between the handle and the adjustment projection. The method may further include inserting the adjustment projection into the drill bore and adjusting a position of the handle thereby causing the drill guide to translate relative the base within the first track opening and the second track opening to adjust the drill guide angle.

According to various embodiments, the method further includes removing the adjustment projection from the drill bore, decoupling the drill guide from the adjustment stand, and providing the drill guide in a desired location proximate a bone. The method may further include rotating the handle while the adjustment projection is positioned within the drill guide causing the drill guide to transition to a locked state to prevent change in the drill guide angle.

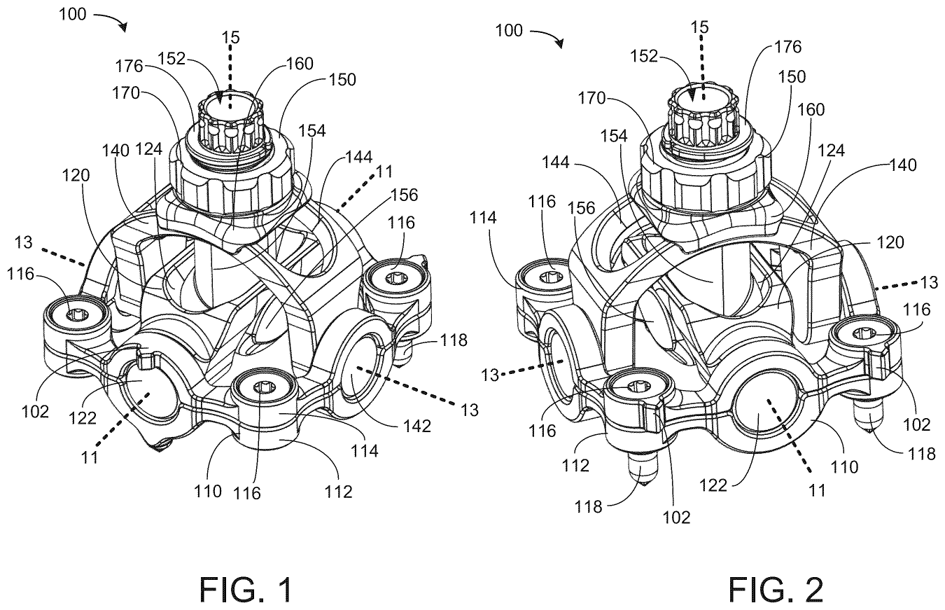

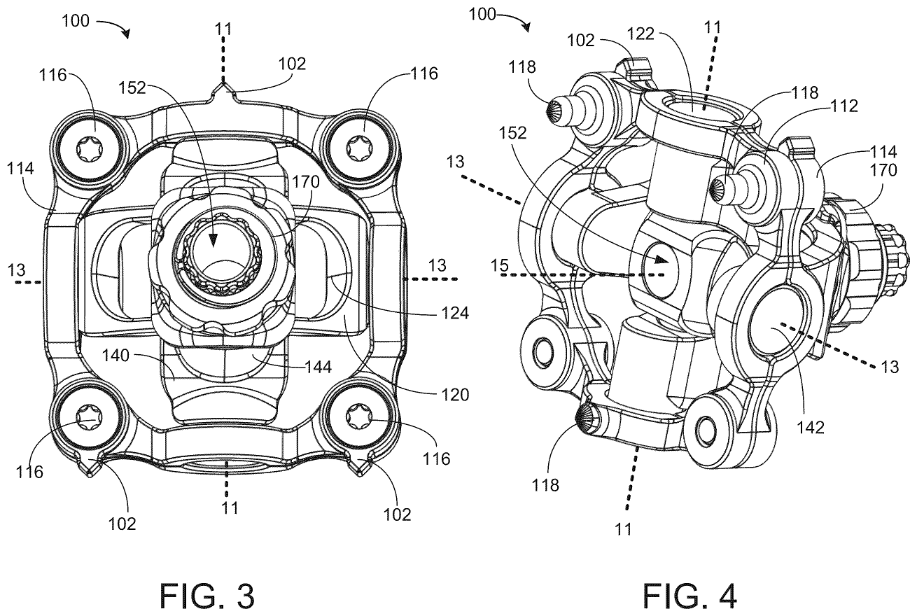

Referring now to FIGS. 1-4, perspective views of a surgical guide 100 are shown, according to an example embodiment. The surgical guide 100 is configured to be adjusted such that a hole can be drilled into a subject at a desired drill angle. The surgical guide 100 includes a base 110 that defines a base plane. For example, the base 110 may include a first foot 118, a second foot 118, and a third foot 118 that define the base plane. As shown, the first foot 118, the second foot 118, and the third foot 118 each come to a pointed foot end. The base plane may be a plane that intersects all three of the pointed foot ends.

The base 110 further includes a first base plate 112 and a second base plate 114 coupled to the first base plate 112 via a plurality of fasteners 116. As is discussed further herein, the first base plate 112 and the second base plate 114 define a plurality of apertures configured to each individually receive a first track projection 122 of a first track 120 of a second track projection 142 of a second track 140, thereby coupling the first track 120 and the second track 140 to the base 110. The second base plate 114 is shown to include a plurality of indicators 102. The indicators 102 may provide visual feedback to a user of the surgical guide 100 to allow them to properly orientate the surgical guide 100.

The surgical guide 100 includes the first track 120. As discussed above, the first track 120 includes a first track projections 122 that are received between the first base plate 112 and the second base plate 114. The first track projections 122 allow the first track 120 to rotate about a first axis 11 relative the base 110. For example, the first track 120 may rotate about the first axis 11 as a user of the surgical guide 100 sets a desired drill guide angle, as is discussed further herein. The first track 120 further includes a first track opening 124 extending through the first track 120. The first track opening 124 is configured to receive a portion of a drill guide 150 and allows a portion of the drill guide 150 to translate within the first track opening 124 as a user of the surgical guide 100 sets a desired drill guide angle. As shown, the first track opening 124 is generally linear in nature. For example, the first track opening 124 may extend in a direction parallel to the base plane.

The surgical guide 100 includes the second track 140. As discussed above, the second track 140 includes a second track projections 142 that are received between the first base plate 112 and the second base plate 114. The second track projections 142 allow the second track 140 to rotate about a second axis 13 relative the base 110. For example, the second track 140 may rotate about the second axis 13 as a user of the surgical guide 100 sets a desired drill guide angle, as is discussed further herein. The first axis 11 and the second axis 13 may be perpendicular one another and/or parallel the base plane. The second track 140 further includes a second track opening 144 extending through the second track 140. The second track opening 144 is configured to receive a portion of a drill guide 150 and allows a portion of the drill guide 150 to translate within the second track opening 144 as a user of the surgical guide 100 sets a desired drill guide angle. As shown, the second track opening 144 is generally arced in nature. For example, the second track opening 144 may extend at a curvature that defines a convex curvature relative the base plane.

The surgical guide 100 further includes a drill guide 150 coupled to the first track 120 and the second track 140. The drill guide 150 includes a drill bore 152 extending along a bore axis 15 from a first end of the drill guide 150 to a second end of the drill guide 150. The drill bore 152 is configured to receive a portion of a drill (e.g., a drill bit), such that a hole can be drilled through the drill bore 152. The drill guide 150 further includes a drill guide body 154 between the first end and the second end of the drill guide 150. The drill guide body 154 is configured to translate within the first track opening 124 and the second track opening as user of the surgical guide 100 sets a desired drill guide angle. The drill guide angle may be defined as the angle formed by the bore axis 15 and the base plane.

The drill guide 150 further includes a locking nut 170. As discussed further herein, the locking nut 170 is configured to be rotated to transition the surgical guide 100 from a locked state to an unlocked state. For example, rotation of the locking nut 170 in a first direction (e.g., relative the bore axis 15) may cause a clamping member 160 to compress against the second track 140 to secure the drill guide 150 in the locked state, such that the drill guide angle is fixed.

The drill guide further includes a retention member 176. The retention member 176 is configured to limit translation of the locking nut 170 along the drill guide 150. For example, the locking nut 170 may be rotated to unlock the drill guide 150. The locking nut 170 may engage the retention member 176 as the locking nut 170 is rotated to or past the unlocked position. Thus, the locking nut 170 may prevent the retention member 176 from being over rotated past the unlocked position, as is discussed further herein.

The drill guide 150 further includes projections 156 extending from the drill guide body 154 proximate the second end of the drill guide 150. As is discussed further herein, the projections 156 are configured to engage the first track 120 to secure the drill guide 150 in the locked state. For example, the projections 156 may compress against the first track 120 (e.g., in a direction opposite the compression of the clamping member 160 against the second track) in response to the locking nut 170 being rotated, to secure the drill guide 150 in the locked state, such that the drill guide angle is fixed.

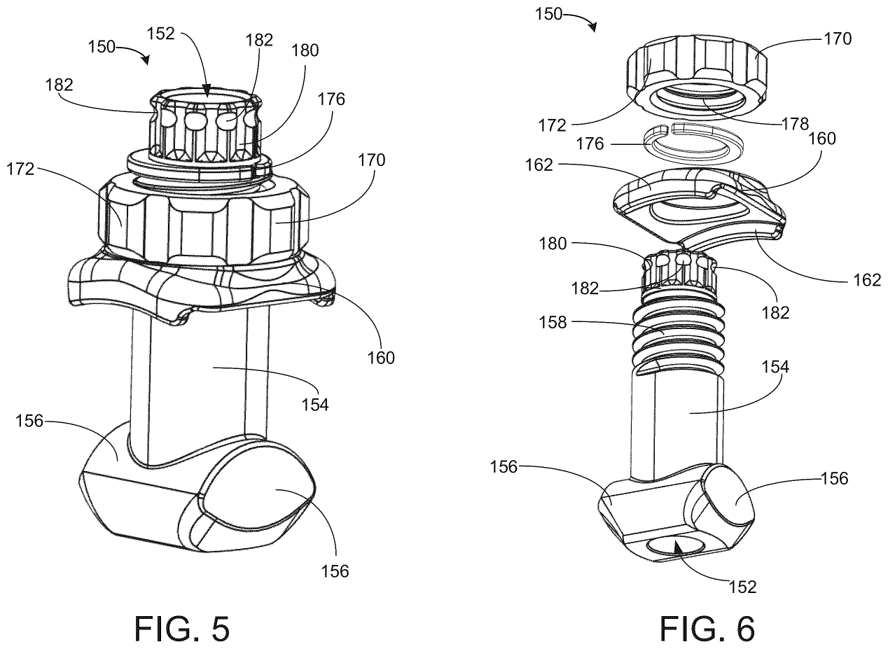

Referring not to FIGS. 5 and 6, a perspective view and an exploded view of the drill guide 150 are shown, respectively, according to an example embodiment. The drill guide 150 includes the drill bore 152 extending through the drill guide body 154. The drill guide body 154 further includes a plurality of threads 158 configured to interface with a plurality of threads 178 on an inner portion of the locking nut 170. As the locking nut 170 is rotated in a first direction, the locking nut 170 translates along the drill guide body 154, thereby compressing the clamping member 160 in a first clamping direction against the second track 140 (see FIGS. 1 and 2). As the locking nut 170 is rotated in a second direction (e.g., opposite the first direction), the drill guide 150 transition to the unlocked state. As the locking nut 170 continues to rotate in the second direction, the locking nut 170 will engage the retention member 176 to prevent over-rotation of the locking nut 170. For example, the retention member 176 may prevent the threads 178 of the locking nut 170 from disengaging the plurality of threads 158 on the drill guide body 154.

Further, the projections 156 may compress against the first track 120 in a second clamping direction, opposite the first clamping direction, in response to the locking nut 170 being rotated in the first direction to secure the drill guide 150 in the locked state, such that the drill guide angle is fixed. The locking nut 170 includes a plurality of splines 172 configured to interface with a plurality of splines (e.g., splines 356 in FIG. 10) to facilitate rotation of the locking nut 170 via an adjustment device.

The clamping member 160 further includes a pair of rails 162 on opposite lateral sides of the clamping member 160. The rails 162 are configured to engage the second track 140 (see FIGS. 1 and 2) as the drill guide 150 translates within the second track opening 144 to restrict relative movement between the drill guide 150 and the second track 140.

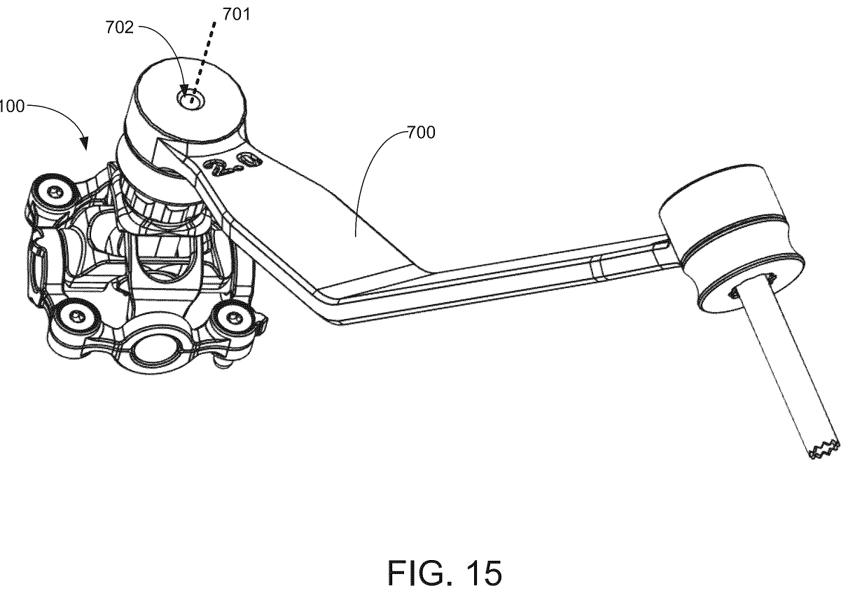

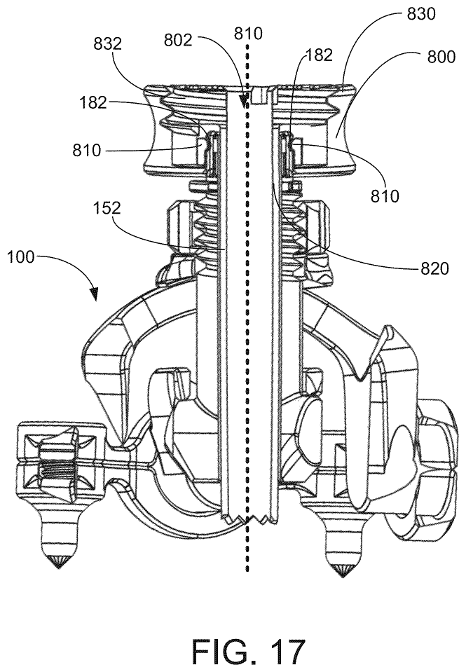

The drill guide 150 further includes an adapter 180 proximate the first end of the drill guide 150. The adapter 180 includes a plurality of splines and is configured to couple the drill guide 150 to a drill guide retainer or a drill guide reducer, as is discussed further with respect to FIGS. 15-17. The adapter 180 further includes a plurality of indents 182 configured to engage one or more projections of a drill guide retainer, as is discussed further below with respect to FIG. 17.

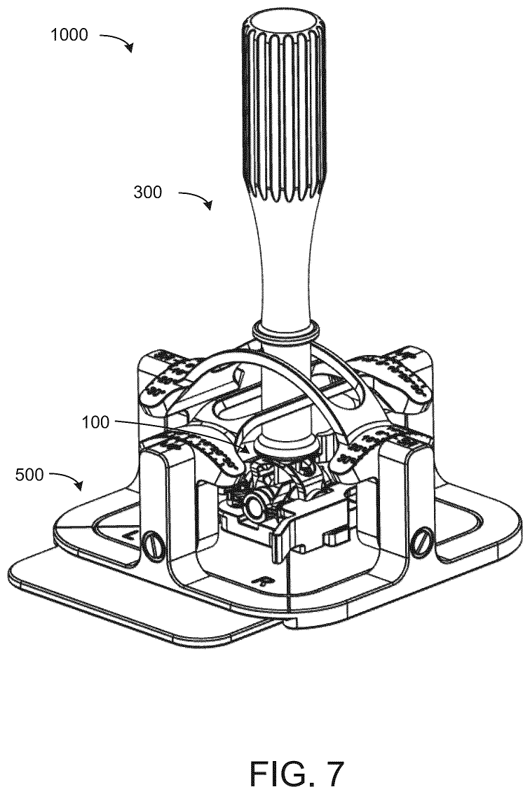

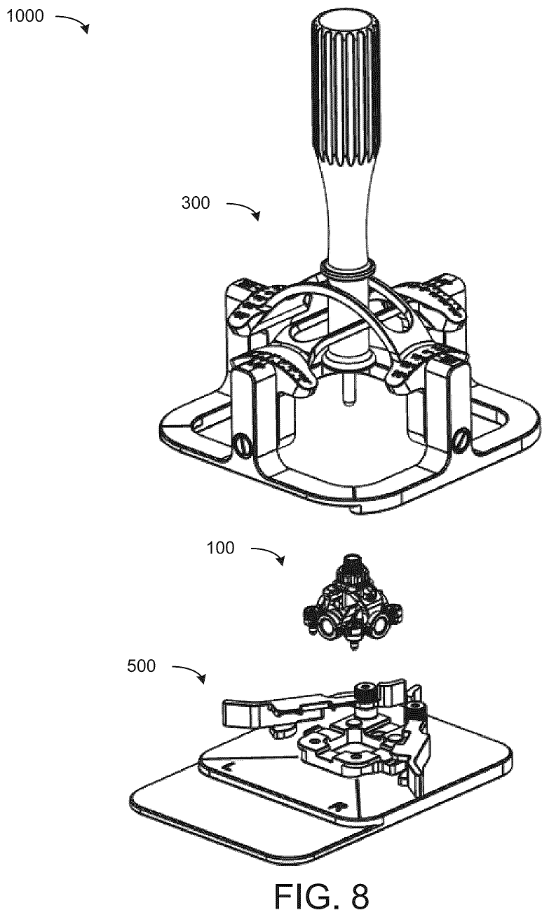

Referring now to FIGS. 7 and 8, a perspective view and an exploded view of a surgical system 1000 are shown, respectively, according to an example embodiment. The surgical system 1000 includes the surgical guide 100, an adjustment device 300, and an adjustment stand 500. As is discussed further herein, the adjustment stand 500 is configured to support (e.g., receive, couple with, etc.) the surgical guide 100. The adjustment stand 500 can then be positioned on top of the adjustment stand 500 such that the surgical guide 100 is positioned between a portion of the adjustment device 300 and a portion of the adjustment stand 500. The adjustment device 300 can then be used to lock and/or unlock the surgical guide 100 and/or adjust a drill guide angle of the drill guide 150.

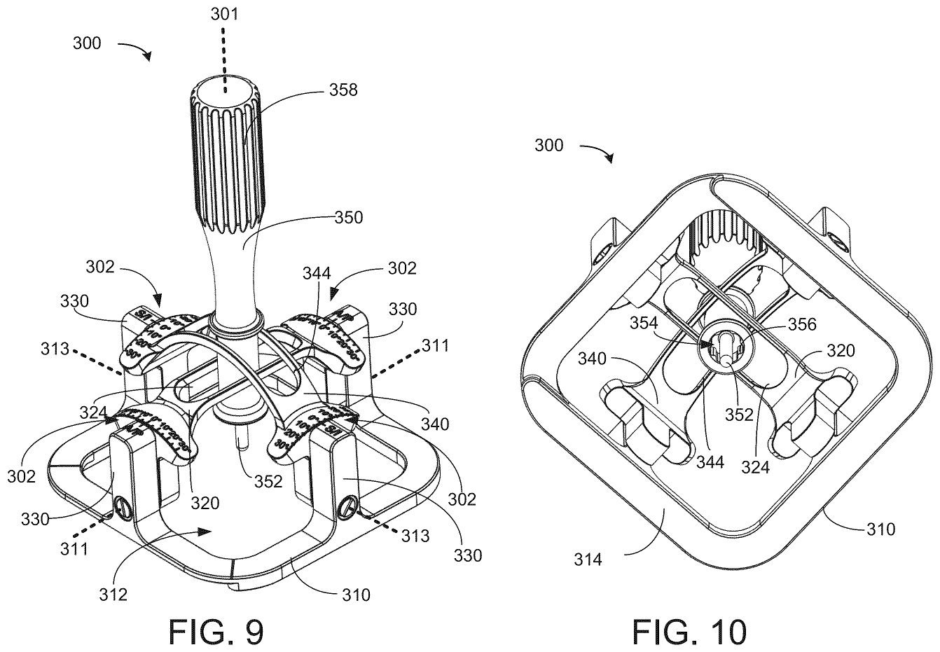

Referring now to FIGS. 9 and 10, perspective views of the adjustment device 300 are shown, according to an example embodiment. The adjustment device 300 is configured to lock or unlock the surgical guide 100. The adjustment device 300 is further configured to adjust a drill guide angle of the surgical guide 100 while the surgical guide 100 is unlocked. The adjustment device 300 includes a base 310 that defines an adjustment base plane. For example, the base 310 may include a bottom surface 314 (see FIG. 10) that defines an adjustment base plane. The base 310 further defines a base cavity 312 configured to receive a portion of the adjustment stand 500 to restrict movement between the adjustment device 300 and the adjustment stand 500 while the adjustment device 300 is positioned on top of the adjustment stand 500.

The adjustment device 300 further includes a plurality of projections 330 coupled to the base 310 and extending away from the bottom surface 314. The projections 330 define a plurality of apertures configured to each individually rotatably couple with a portion of a first adjustment track 320 or a second adjustment track 340, thereby coupling the first adjustment track 320 and the second adjustment track 340 to the base 310. The first adjustment track 320 is configured to rotate to rotate about a first axis 311 relative the base 310. For example, the first adjustment track 320 may rotate about the first axis 311 as a user of the adjustment device 300 sets a desired drill guide angle.

The first adjustment track 320 further includes a first adjustment track opening 324 extending through the first adjustment track 320. The first adjustment track opening 324 is configured to receive a portion of a drill guide adjuster 350 and allows a portion of the drill guide adjuster 350 to translate within the first adjustment track opening 324 as a user of the adjustment device 300 sets a desired drill guide angle. As shown, the first adjustment track opening 324 is generally linear in nature. For example, the first adjustment track opening 324 may extend in a direction parallel to the adjustment base plane.

The adjustment device 300 includes the second adjustment track 340. The second adjustment track 340 is rotatably coupled to two of the projections 330 and is configured to rotate about a second axis 313 relative the base 310. For example, the second adjustment track 340 may rotate about the second axis 313 as a user of the adjustment device 300 sets a desired drill guide angle, as is discussed further herein. The first axis 311 and the second axis 313 may be perpendicular one another and/or parallel the adjustment base plane. The second adjustment track 340 further includes a second adjustment track opening 344 extending through the second adjustment track 340. The second adjustment track opening 344 is configured to receive a portion of a drill guide adjuster 350 and allows a portion of the drill guide adjuster 350 to translate within the second adjustment track opening 344 as a user of the adjustment device 300 sets a desired drill guide angle. As shown, the second adjustment track opening 344 is generally arced in nature. For example, the second adjustment track opening 344 may extend at a curvature that defines a convex curvature relative the adjustment base plane.

The adjustment device 300 is shown to include a plurality of angular indicators 302. The angular indicators 302 may provide visual feedback to a user of the adjustment device 300 to allow them to properly orientate the drill guide angle of the surgical guide 100. For example, the angular indicators 302 may correspond with the drill guide angle of the surgical guide 100. For example, a first set of angular indicators 302 may indicate an angle of the drill guide 150 relative the base 110 within a first adjustment plane (e.g., corresponding with an angular orientation of the first track 120). Further, a second set of angular indicators 302 may indicate an angle of the drill guide 150 relative the based within a second adjustment plane (e.g., corresponding with an angular orientation of the second track 140).

The adjustment device 300 further includes the drill guide adjuster 350 coupled to the first adjustment track 320 and the second adjustment track 340. The drill guide adjuster 350 includes a handle 358 proximate a first end of the drill guide adjuster 350 and configured to be manipulated by a user of the adjustment device 300 as a user of the adjustment device 300 sets a desired drill guide angle.

Referring to FIG. 10, the drill guide adjuster 350 further includes an adjustment projection 352 proximate a second end of the drill guide adjuster 350. The adjustment projection 352 is configured to be inserted into the drill bore 152 (see FIGS. 1 and 2) such that manipulation of the handle 358 causes a change in the drill guide angle. The adjustment projection 352 extends from within an adjustment cavity 354 defined by the drill guide adjuster 350 proximate the second end of the drill guide adjuster 350. The adjustment cavity 354 is configured to receive the locking nut 170 (see FIG. 5) when the adjustment device 300 is positioned on top of the surgical guide 100. As the handle 358 is rotated, a plurality of splines 356 interface with the plurality of splines 172, thereby causing the locking nut 170 to rotate in response to the handle 358 being rotated to lock or unlock the surgical guide 100.

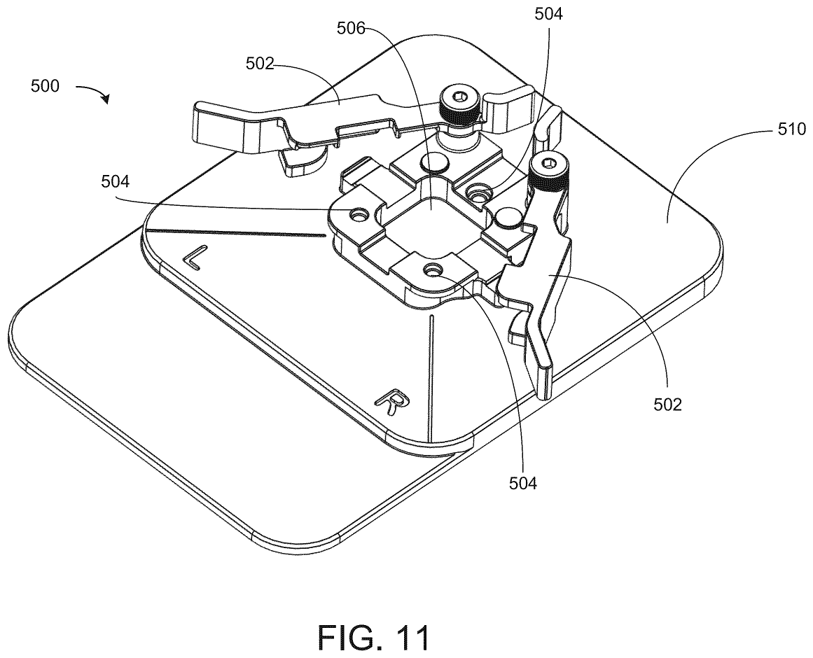

Referring now to FIG. 11, a perspective view of the adjustment stand 500 is shown, according to an example embodiment. The adjustment stand 500 is configured to receive and secure the adjustment device 300. As shown, the adjustment device 300 includes a stand base 510. The stand base 510 is configured to be positioned within the base cavity 312 of the adjustment device 300 to restrict relative movement between the adjustment stand 500 and the adjustment device 300.

The adjustment stand 500 further includes a guide cavity 506 defined by the stand base 510. The guide cavity 506 is configured to receive the adjustment device 300. The adjustment stand 500 further includes feet cavities 504 defined by the stand base 510. The feet cavities 504 are configured to individually receive the first foot 118, the second foot 118, and the third foot 118 (see FIG. 4) to restrict movement between the adjustment stand 500 and the surgical guide 100 while the surgical guide 100 is positioned within the guide cavity 506. Further, the adjustment stand 500 includes arms 502 configured to rotate to secure the surgical guide 100 to the adjustment stand 500 as discussed further herein.

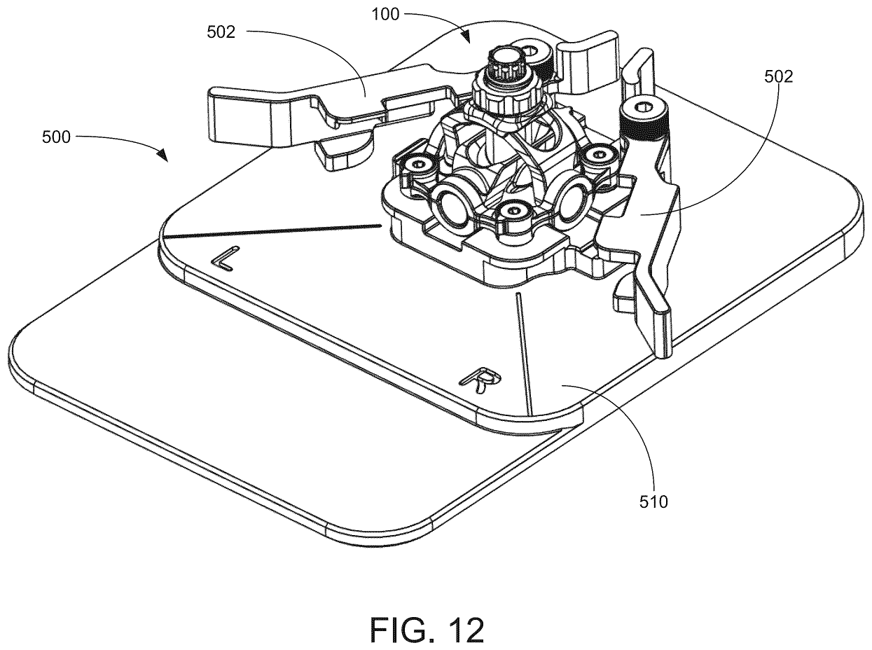

Referring now to FIG. 12, a perspective view of the surgical guide 100 positioned on top of the adjustment stand 500, according to an example embodiment. The arms 502 are in an open state such that the surgical guide 100 can placed within the guide cavity 506 and/or removed from the guide cavity 506.

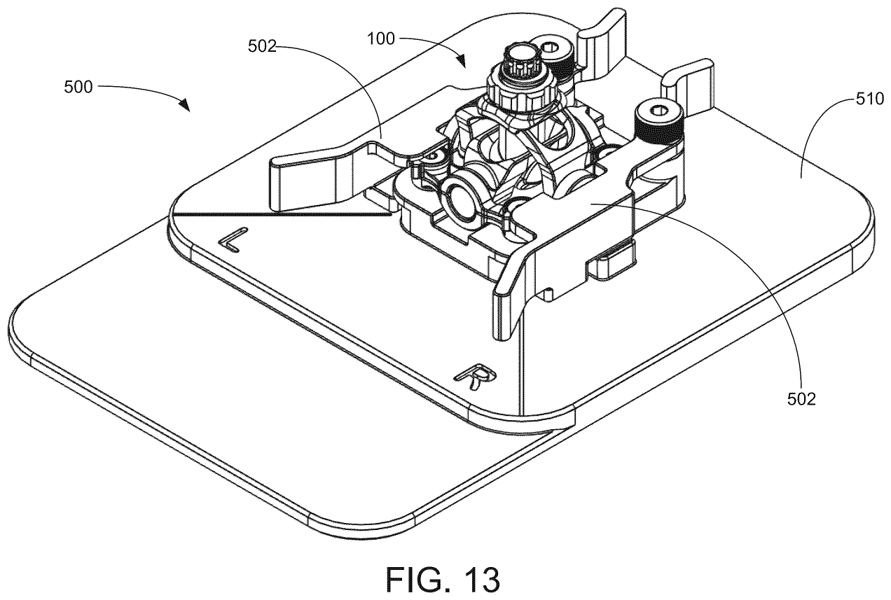

Referring now to FIG. 13 is a perspective view of the surgical guide of 100 coupled to the adjustment stand 500 is shown, according to an example embodiment. The arms 502 are in a closed state to restrict movement of the surgical guide 100 relative the adjustment stand 500. After the surgical guide 100 is coupled to the adjustment stand 500, the adjustment device 300 can be placed on top of the adjustment stand 500 and the adjustment projection 352 can be inserted into the drill bore 152 such that the adjustment device 300 can be used to adjust the drill guide angle of the surgical guide 100.

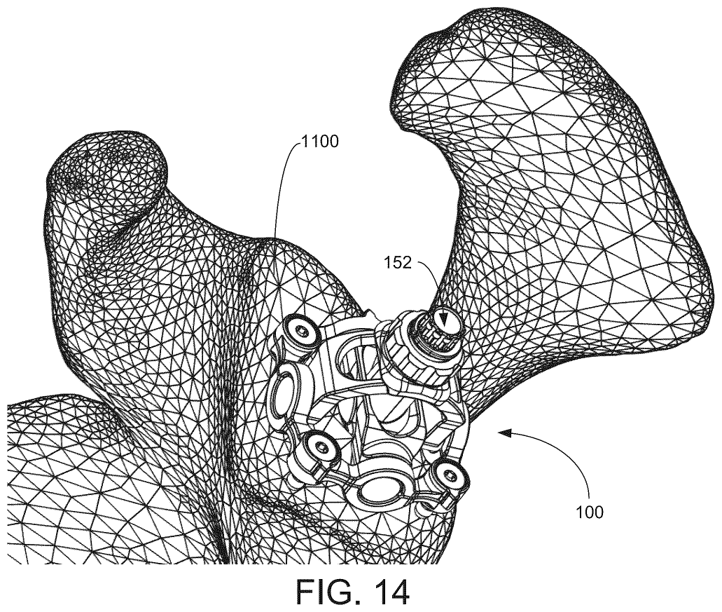

Referring now to FIG. 14, a perspective view of the surgical guide 100 positioned proximate a tissue 1100 is shown, according to an example embodiment. For example, the tissue 1100 may be a part of a shoulder bone and the surgical guide 100 may be positioned proximate the glenoid. The drill guide angle of the surgical guide 100 may be set at a desired angle such that a hole can be drilled through the drill bore 152 a predetermined angle (e.g., an optimal drill angle). For example, the surgical guide 100 may be used during a shoulder arthroplasty or a shoulder replacement surgery.

Referring now to FIG. 15 is a perspective view of a drill guide retainer 700 (e.g., a drill guide with a handle) coupled to the surgical guide 100 is shown, according to an example embodiment. As discussed further herein, the surgical guide 100 includes an adapter 180 (see FIG. 5) configured to couple the drill guide to the drill guide retainer 700. The drill guide retainer 700 includes a retainer opening 702 extending along a retainer opening axis 701. The retainer opening axis 701 may alight with the bore axis 15 (see FIGS. 1 and 2) when the drill guide retainer 700 is coupled to the surgical guide 100. The drill guide retainer is configured to further guide the drill bit through the retainer opening and the drill bore 152 (see FIGS. 1 and 2) to create a hole in tissue of a subject. Further, the drill guide retainer 700 may reduce a cross section of the drill bore 152 such that a smaller diameter hole can be drilled using the surgical guide 100. As shown, the drill guide retainer 700 is a 2.0 mm drill guide retainer. It should be appreciated that other size drill guide retainers may be used (e.g., 1 mm, 1.5 mm, 2.5 mm, 3 mm, 3.5 mm, etc.).

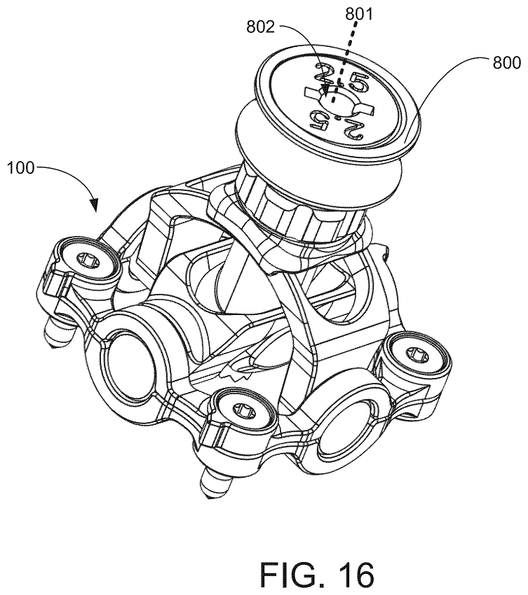

Referring now to FIG. 16 is a perspective view of a drill guide retainer 800 (e.g., a drill guide without a handle) coupled to the surgical guide 100 is shown, according to an example embodiment. As discussed further herein, the surgical guide 100 includes an adapter 180 (see FIG. 5) configured to couple the drill guide to the drill guide retainer 800. The drill guide retainer 800 includes a retainer opening 802 extending along a retainer opening axis 801. The retainer opening axis 801 may alight with the bore axis 15 (see FIGS. 1 and 2) when the drill guide retainer 800 is coupled to the surgical guide 100. The drill guide retainer is configured to further guide the drill bit through the retainer opening and the drill bore 152 (see FIGS. 1 and 2) to create a hole in tissue of a subject. Further, the drill guide retainer 800 may reduce a cross section of the drill bore 152 such that a smaller diameter hole can be drilled using the surgical guide 100. As shown, the drill guide retainer 800 is a 2.5 mm drill guide retainer. It should be appreciated that other size drill guide retainers may be used (e.g., 1 mm, 1.5 mm, 2.0 mm, 3 mm, 3.5 mm, etc.). While FIGS. 15 and 16 show the surgical guide 100 being used with a drill guide retainer, it should be appreciated that the surgical guide 100 may be used as a drill guide without a drill guide retainer.

Referring now to FIG. 17, a cross sectional view of the drill guide retainer 800 coupled to the surgical guide 100 is shown, according to an example embodiment. The surgical guide 100 includes the indents 182 configured to individually receive a projection 810 of the drill guide retainer 800, thereby coupling the drill guide reducer to the surgical guide 100. For example, the interaction between the indents 182 and the projections 810 may create a snap fit between the drill guide retainer 800 and the indents 182.

According to the example embodiment shown, the drill guide reducer 800 includes a drill guide shaft 820 coupled to an outer ring 830. The drill guide shaft 820 is configured to be inserted into the drill guide bore 152 such that a hole can be drilled through the drill guide shaft 820. As discussed above, the outer ring 830 includes the projections 810 that create a snap fit between the drill guide retainer 800 and the indents 182. Further, the outer ring 830 includes threads 802 configured to couple with cannula threads.

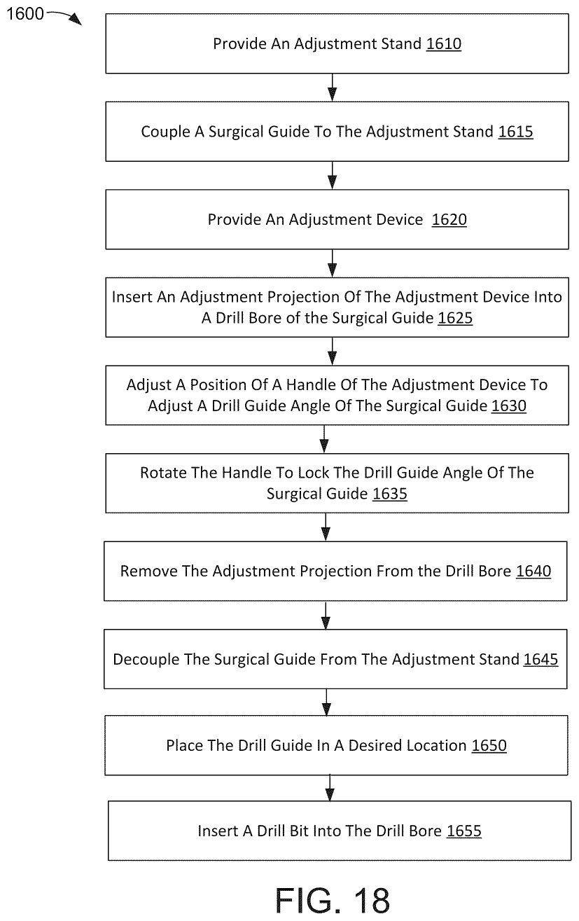

Referring now to FIG. 18 is a block diagram illustrating a method of using the surgical guide 1600 is shown, according to an example embodiment. The method 1600 may utilize one or more of the components or devices described herein. It should be appreciated that the steps need not be performed in the order shown. Further, various steps may be omitted, and additional steps may be performed.

At step 1610, an adjustment stand is provided. For example, the adjustment stand 500 may be provided onto a flat surface. The adjustment stand 500 may be provided with the arms 502 in the open state (e.g., as shown in FIG. 11) such that a surgical guide may be placed on top of the adjustment stand.

At step 1615, a surgical guide is coupled to the adjustment stand. For example, the surgical guide 100 may be coupled to the adjustment stand 500. The surgical guide 100 may be placed on top of the adjustment stand 500 within the guide cavity 506 and the feet 118 may be positioned within the feet cavities 504 (e.g., as shown in FIG. 12). The arms 502 of the adjustment stand 500 may then be rotated to a closed state to couple the surgical guide 100 to the adjustment stand 500 (e.g., as shown in FIG. 13)

At step 1620, an adjustment device is provided. For example, the adjustment device 300 may be provided above the adjustment stand (e.g., as shown in FIG. 8).

At step 1625, an adjustment projection of the adjustment device is inserted into a drill bore of the surgical guide. For example, the adjustment projection 352 of the adjustment device 300 may be provided within the drill bore 152 of the surgical guide 100 (e.g., as shown in in FIG. 7). Further, step 1625 may include positioning the stand base 510 within the base cavity 312 of the adjustment device 300 to restrict relative movement between the adjustment stand 500 and the adjustment device 300.

At step 1630, a position of a handle of the adjustment device is adjusted to adjust a drill guide angle of the surgical guide. For example, a user of the adjustment device 300 may manipulate the handle 358, thereby causing a change in the drill guide angle. The user may utilize the angular indicators 302 to provide visual feedback to the user of the adjustment device 300 to allow the user to properly orientate the drill guide angle of the surgical guide 100. For example, the angular indicators 302 may correspond with the drill guide angle of the surgical guide 100. It should be appreciated that step 1630 may further involve rotating the handle 358 to transition the surgical guide 100 to the unlocked state.

At step 1635, the handle of the adjustment device is rotated to lock the drill guide angle of the surgical guide. For example, the handle 358 may be rotated, thereby causing the locking nut 170 to rotate in response to the handle 358 being rotated to lock the surgical guide 100. Once the surgical guide is in the locked state, the drill guide angle may be fixed.

At step 1640, the adjustment projection is removed from the drill bore. For example, the adjustment device 300 may be pulled off the top of the surgical guide, thereby removing the adjustment projection 352 from the drill bore 152.

At step 1645, the surgical guide is decoupled from the adjustment stand. For example, the arms 502 of the adjustment stand 500 may then be rotated to an open state such that the surgical guide 100 can be removed from the adjustment stand 500.

At step 1650 the drill guide is placed in a desired location. For example, the surgical guide 100 may be positioned proximate the tissue 1100 (e.g., as shown in FIG. 14).

At step 1655, a drill bit is inserted into the drill bore. For example, a drill bit may be inserted into the drill bore 152 and a hole may be drilled in the tissue 1100.

### B. Virtual Surgical Planning

The surgical guide described in Section A can be used by physicians in surgical procedures. Physicians or clinicians often engage in surgical planning before performing surgery, frequently using virtual surgical planning software, which allows for the virtual planning and preparation of surgical procedures. The present disclosure describes systems and methods for integrating the use of a surgical guide within the virtual planning environment. In particular, the present disclosure provides a process for generating a virtual model of the surgical guide from Section A and deploying the virtual model into the virtual surgical planning software. This enables surgeons to plan surgeries with the aid of the surgical guide.

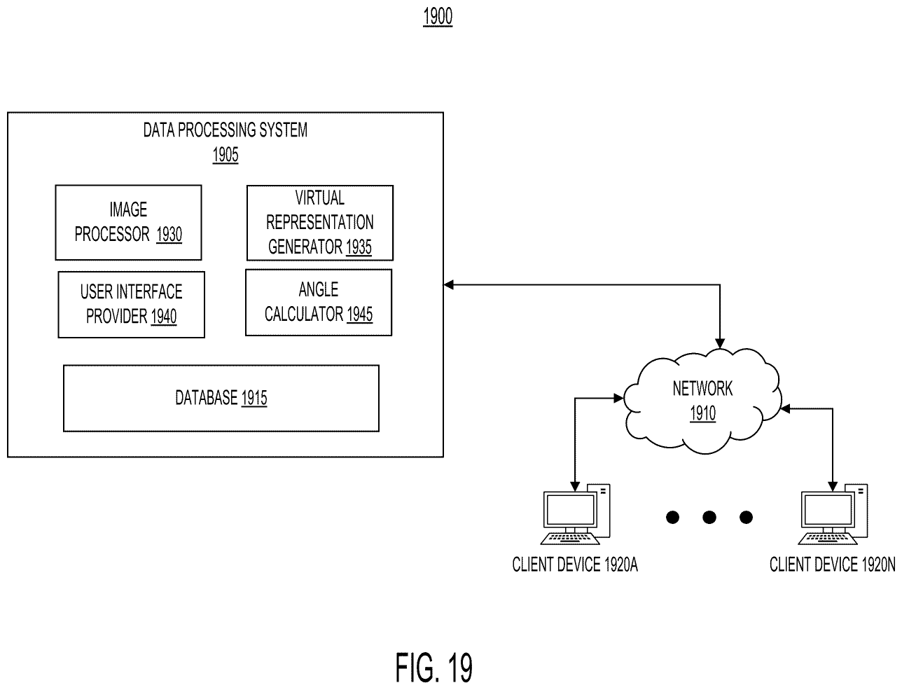

Referring now to FIG. 19, depicted is a block diagram of an example system 1900 for providing virtual surgical planning functionalities, in accordance with one or more implementations. The system 1900A can include at least one data processing system 1905, at least one network 1910, and one or more client devices 1920A-1920N (sometimes generally referred to as client device(s) 1920).

As shown in FIG. 19, the data processing system 1905 can include at least one image processor 1930, at least one virtual representation generator 1935, at least one user interface provider 1940, at least one angle calculator 1945, and at least one database 1915. Each of the components (e.g., the data processing system 1905, the network 1910, the client devices 1920, etc.) of the system 1900 can be implemented using the hardware components or a combination of software with the hardware components of a computing system, such as the server 2600 or the client computing system 2614 described in connection with FIG. 26, or any other computing system described herein.

The data processing system 1905 can include at least one processor and a memory, e.g., a processing circuit. The memory can store processor-executable instructions that, when executed by processor, cause the processor to perform one or more of the operations described herein. The processor may include a microprocessor, an application-specific integrated circuit (ASIC), a field-programmable gate array (FPGA), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The data processing system 1905 can include one or more computing devices or servers that can perform various functions as described herein. The data processing system 1905 can include any or all of the components and perform any or all of the functions of a server system 2600 described herein in conjunction with FIG. 26.

The network 1910 can include computer networks such as the Internet, local, wide, metro or other area networks, intranets, satellite networks, other computer networks such as voice or data mobile phone communication networks, and combinations thereof. The data processing system 1905 of the system 1900A can communicate via the network 1910, for example with one or more client devices 1920. The network 1910 may be any form of computer network that can relay information between the data processing system 1905, the one or more client devices 1920, and one or more information sources, such as web servers or external databases, amongst others. In some implementations, the network 1910 may include the Internet and/or other types of data networks, such as a local area network (LAN), a wide area network (WAN), a cellular network, a satellite network, or other types of data networks.

The network 1910 may also include any number of computing devices (e.g., computers, servers, routers, network switches, etc.) that are configured to receive and/or transmit data within the network 1910. The network 1910 may further include any number of hardwired and/or wireless connections. Any or all of the computing devices described herein (e.g., the data processing system 1905, the one or more client devices 1920, the server system 2600, the client computing system 2614, etc.) may communicate wirelessly (e.g., via Wi-Fi, cellular, radio, etc.) with a transceiver that is hardwired (e.g., via a fiber optic cable, a CAT5 cable, etc.) to other computing devices in the network 1910. Any or all of the computing devices described herein (e.g., the data processing system 1905, the one or more client devices 1920, the server system 2600, the client computing system 2614, etc.) may also communicate wirelessly with the computing devices of the network 1910 via a proxy device (e.g., a router, network switch, or gateway).

Each of the client devices 1920 can include at least one processor and a memory, e.g., a processing circuit. The memory can store processor-executable instructions that, when executed by processor, cause the processor to perform one or more of the operations described herein. The processor can include a microprocessor, an ASIC, an FPGA, etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions can include code from any suitable computer programming language. The client devices 1920 can include one or more computing devices or servers that can perform various functions as described herein. The one or more client devices 1920 can include any or all of the components and perform any or all of the functions of the client computing system 2614 described herein in conjunction with FIG. 26.

Each client device 1920 can include, but is not limited to, a mobile device (e.g., a smartphone, tablet, etc.), a television device (e.g., smart television, set-top box, et.), a personal computing device (e.g., a desktop, a laptop, etc.) or another type of computing device. Each client device 1920 can be implemented using hardware or a combination of software and hardware. Each client device 1920 can include a display or display portion. The display can include a display portion of a television, a display portion of a computing device, or another type of interactive display (e.g., a touchscreen, a display, etc.) and one or more input/output (I/O) devices (e.g., a mouse, a keyboard, digital keypad).

The display can include a touch screen displaying an application, such as the gaming applications described herein. The display can include a border region (e.g., side border, top border, bottom border). In some implementations, the display can include a touch screen display, which can receive interactions from a player. The interactions can result in interaction data, which can be stored and transmitted by the processing circuitry of the client device 1920. The interaction data can include, for example, interaction coordinates, an interaction type (e.g., click, swipe, scroll, tap, etc.), and an indication of an actionable object with which the interaction occurred.

The client application can include an application executing on each client device 1920 or provided to the client device 1920 by the system 1900. The application can include a web application, a server application, a resource, a desktop, or a file. In some implementations, the application can include a local application (e.g., local to a client device 1920), hosted application, Software as a Service (SaaS) application, virtual application, mobile application, and other forms of content. In some implementations, the application can include or correspond to applications provided by remote servers or third-party servers.

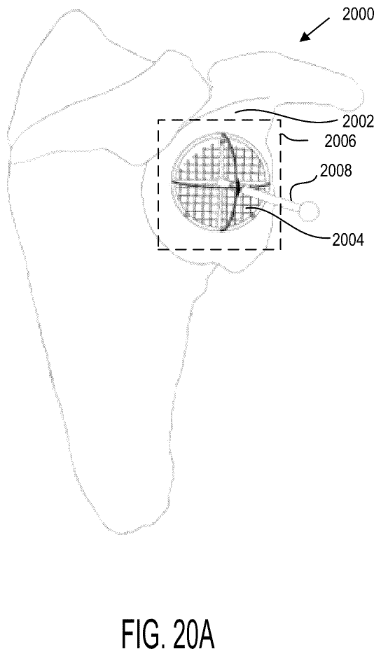

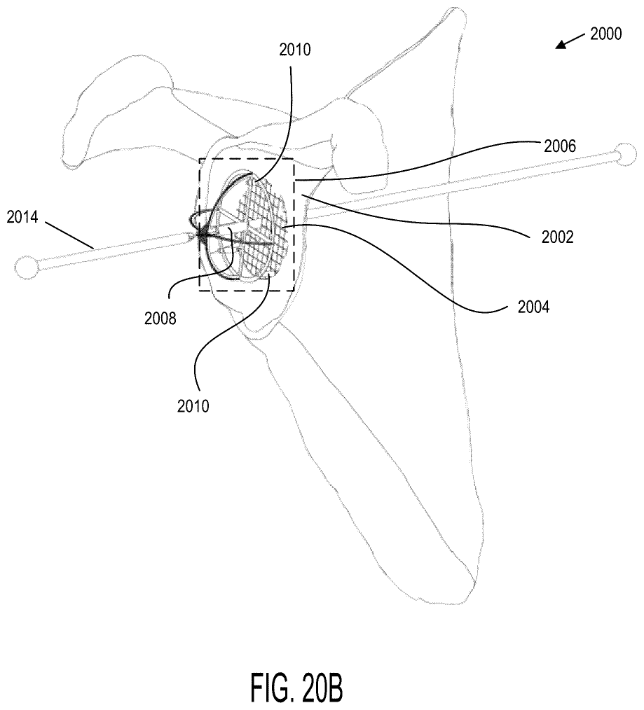

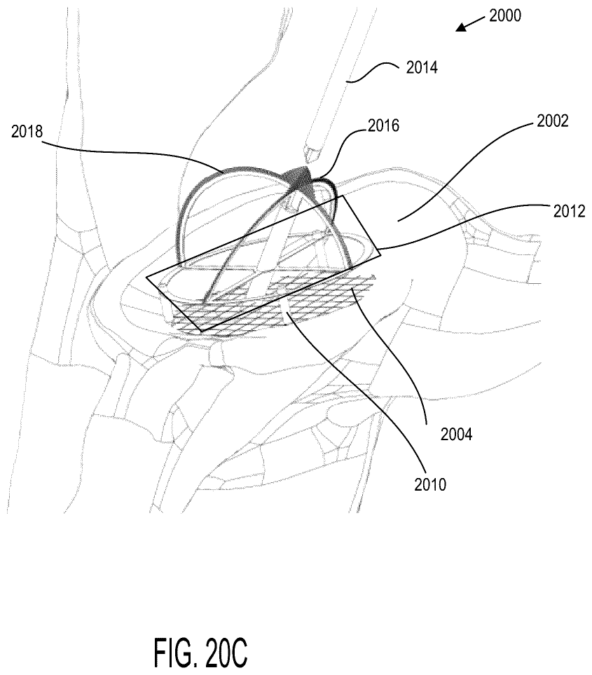

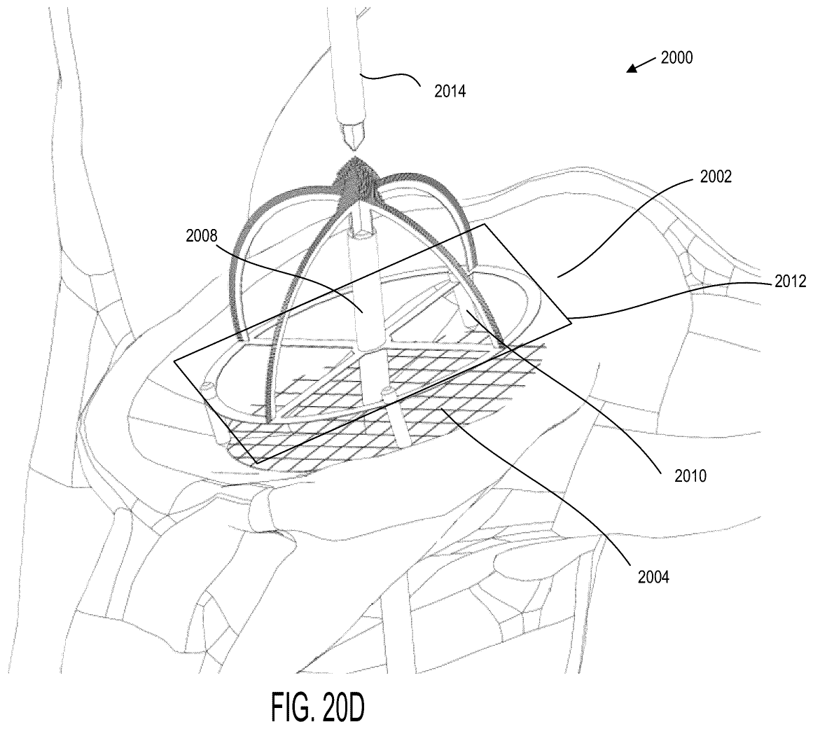

In some implementations, one or more client devices 1920 can establish one or more communication sessions with the data processing system 1905. The one or more communication sessions can each include a channel or connection between the data processing system 1905 and the one or more client devices 1920. The one or more communication systems can each include an application session (e.g., virtual application), an execution session, a desktop session, a hosted desktop session, a terminal services session, a browser session, a remote desktop session, a URL session and/or a remote application session. Each communication session can include encrypted and/or secure sessions, which can include an encrypted file, encrypted data or traffic. The client devices 1920 can each use the communication session established with the data processing system 1905 to carry out any of the functionalities described herein. For example, the application executing on a client device 1920 can perform any of the client-side operations described herein, including displaying any of the user interfaces shown in FIGS. 20A-20D, or any other types of user interfaces described herein.

The data processing system 1905 is shown as including the database 1915. The database 1915 can be a computer-readable memory that can store or maintain any of the information described herein. The database 1915 can maintain one or more data structures, which may contain, index, or otherwise store each of the values, pluralities, sets, variables, vectors, numbers, or thresholds described herein. The database 1915 can be accessed using one or more memory addresses, index values, or identifiers of any item, structure, or region maintained in the database 1915. The database 1915 can be accessed by the components of the data processing system 1905, or any other computing device described herein, via the network 1910. In some implementations, the database 1915 can be internal to the data processing system 1905. In some implementations, the database 1915 can exist external to the data processing system 1905 and may be accessed via the network 1910. For example, the database 1915 may be distributed across many different computer systems (e.g., a cloud computing system) or storage elements and may be accessed via the network 1910 or a suitable computer bus interface.

The data processing system 1905 can store, in one or more regions of the memory of the data processing system 1905, or in the database 1915, the results of any or all computations, determinations, selections, identifications, generations, constructions, or calculations in one or more data structures indexed or identified with appropriate values. Any or all values stored in the database 1915 may be accessed by any computing device described herein, such as the data processing system 1905, to perform any of the functionalities or functions described herein. In implementations where the database 1915 forms a part of a cloud computing system, the database 1915 can be a distributed storage medium in a cloud computing system and can be accessed by any of the components of the data processing system 1905, by the one or more client devices 1920 (e.g., via the user interface similar to that depicted in FIGS. 20A-20D, etc.), or any other computing devices described herein.

The database 1915 can include information to facilitate virtual surgical planning. For example, for patient-specific data, the database 1915 can include various fields, including, but not limited to, demographics, medical history, and imaging data, among others. Additionally, for surgical guide design data, the database 1915 can include records for surgical guide models, including their mechanical properties and compatible surgical implants. In certain implementations, the database 1915 can maintain a virtual model of a surgical guide within a data structure, which may include a combination of associated attributes to represent the surgical guide's shape and size. Similarly, regarding surgical procedure data, the database can include records of established protocols.

Referring now to the operations of the data processing system 1905, the data processing system 1905 can include an image processor 1930, which can be a script, module, library, or function that receives and processes preoperative image data of a glenoid to identify predefined anatomical landmarks within the image data. In certain implementations, the image processor 1930 can receive various types of medical imaging formats, such as X-rays and CT scans, to identify a glenoid. In certain implementations, the image processor 1930 can process the received image data to identify the predefined anatomical landmarks, including, but not limited to, the scapula, the humeral head, and other landmarks associated with the glenoid.

The data processing system 1905 can include a virtual representation generator 1935, which can be a script, module, library, or function that generates a virtual representation of a glenoid face based on the predefined anatomical landmarks identified in the preoperative image data. In certain implementations, the virtual representation generator 1935 can generate a virtual boundary on a virtual surface of the glenoid face. In certain implementations, the virtual boundary can be dynamically adjustable. The virtual boundary can cover the relevant portion of the glenoid face targeted for surgical intervention. In certain implementations, the adaptability of the virtual boundary can be customized to fit a patient's specific anatomy.

The data processing system 1905 can include an interface provider 1940, which can be a script, module, library, or function that provides a user interface. In certain implementations, the interface provider 1940 can facilitate the visualization and analysis of a surgical guide within a virtual environment. In certain implementations, the interface provider 1940 can present a virtual representation of the glenoid face. Additionally, the interface provider 1940 can present a virtual model of the surgical guide.

The interface provider 1940 can provide one or more interactive elements that a user can interact with. The interactive elements can include, but are not limited to, interactive buttons, voice commands, joysticks (or other tactile devices), gesture recognition systems, or other haptic devices providing haptic feedback through vibrations or forces. The interactive buttons can enable users to initiate specific actions, such as rotating or anchoring the virtual guide model. For hands-free control, the data processing system 1905 can receive and interpret vocal instructions. The gesture recognition systems can enable the user to control the virtual model of the surgical guide through natural hand movements.

Upon interacting with the interactive element, the interface provider 1940 can facilitate the dynamic alignment of the virtual model of the surgical guide with the virtual representation of the glenoid face. In certain implementations, the interface provider 1940 may restrict (via the data processing system 1905) the positioning or orientation of the virtual model of the surgical guide within a virtual boundary on the glenoid. In certain implementations, when the virtual model of the surgical guide extends beyond the virtual boundary, the data processing system 1905 can cause the interface provider 1940 to present informative feedback, such as error messages or visual indicators.

The virtual model of the surgical guide can define a virtual base plane that can be defined by a number of virtual prongs on the surgical guide. The virtual prongs are to be positioned on the virtual surface of the glenoid face within the defined virtual boundary. In certain implementations, the data processing system 1905 can automatically position the virtual prongs at defined locations on the glenoid. For example, the data processing system 1905 can use predefined rules based on known anatomical landmarks on the glenoid to determine optimal locations for the virtual prongs. In certain implementations, the data processing system 1905 can automatically position the virtual prongs based on a specific geometric pattern (e.g., triangle, square) to provide distributed support for the surgical guide within the defined virtual boundary. In certain implementations, the user can initially place one or two virtual prongs on the virtual surface of the glenoid face at the desired location. The data processing system 1905 can use the initial placement and anatomical information to automatically position the remaining prongs (or all the prongs in certain implementations).

In certain implementations, the virtual model of the surgical guide can use its shape or features as markers for validation. For example, the virtual model of the surgical guide can validate its precise positioning (or corresponding angles as described herein) by aligning or matching its shape or features with the target anatomy (e.g., glenoid) within the virtual environment, thereby confirming or indicating via the interface provider 1940 that the surgical guide is correctly or accurately positioned or aligned with the intended surgical site. For example, when the edges or virtual prongs of the virtual model of the surgical guide align with the virtual representation of the glenoid, the surgical guide can indicate correct positioning on the bone. In certain implementations, the virtual model of the surgical guide can use or incorporate additional points beyond the initial three virtual prongs to increase the accuracy of its placement. For example, by comparing or aligning the extra points with the anatomy of the glenoid or scapula, the surgical guide can validate via the data processing system 1905 that the surgical intervention targets the correct anatomical locations.

In certain implementations, if the user adjusts the virtual model of the surgical guide, the data processing system 1905 can automatically reposition the virtual prongs to new locations on the glenoid. The data processing system 1905 can reposition the virtual prongs based on anatomical constraints and the virtual boundary. For example, upon identifying a new target area on the glenoid, the data processing system 1905 can determine new positions for the virtual prongs to align with the new target area, considering the virtual boundary that marks the safe area on the glenoid.

In certain implementations, the interactive provider 1940 can provide feedback to indicate alignment confirmation. In certain implementations, the interface provider 1940 can provide visual cues to indicate alignment, such as highlighting corresponding anatomical landmarks or displaying overlapping planes. In certain implementations, the data processing system 1905 can differentiate between soft touch (initial contact) and firm contact (stable placement) with visual, haptic, or auditory feedback.

The data processing system 1905 can include an angle calculator 1945, which can be a script, module, library, or function that calculates a version angle and an inclination angle of the surgical guide relative to a glenoid plane. The glenoid plane may be referred to as the two-dimensional geometric plane that best fits the virtual surface of the glenoid face. Upon aligning the virtual model of the surgical guide with the virtual representation of the glenoid face, the angle calculator 1945 can determine the version angle, indicating a backward or forward tilt of the surgical guide's axis relative to the glenoid plane, and the inclination angle, indicating the downward or upward tilt of the surgical guide's axis relative to the glenoid plane. The axis of the surgical guide can refer to an imaginary line traversing/running through the center of the surgical guide. The axis of the surgical guide can be a reference point for determining the orientation of the surgical guide relative to the glenoid plane.

In certain implementations, the angle calculator 1945 can determine the angles while the user is adjusting the position and orientation of the virtual model of the surgical guide. In certain implementations, the angle calculator 1945 can determine the angles based on the alignment of the virtual base plane, defined by the number of virtual prongs of the surgical guide. The angle calculator 1945 can establish coordinate systems for the glenoid plane and virtual base plane. The coordinate systems can provide a framework for mathematical calculations. For example, the version angle can be derived by calculating the angle between the X-axes of the glenoid plane and the virtual base plane. A positive angle can indicate a backward tilt, and a negative angle can indicate a forward tilt of the surgical guide relative to the glenoid plane, or vice versa. Additionally, the inclination angle can be derived by calculating the angle between the Z-axes of the glenoid plane and the virtual base plane. A positive angle can indicate a downward tilt, and a negative angle can indicate an upward tilt of the surgical guide relative to the glenoid plane, or vice versa.

Referring now to FIGS. 20A-20H in the context of the components described in connection with FIG. 1, a user interface 2000 is presented on a client device 1920. As shown in FIGS. 20A-20H, the user interface 2000 presents one or more perspective views of a virtual model of a surgical guide 2008 in conjunction with a virtual representation of a glenoid. As depicted in FIG. 20A, the user interface 2000 presents a virtual representation of a patient's glenoid face 2002 generated from preproperating imaging data (CT scans, X-rays, etc.). In certain implementations, the virtual representation of the glenoid face 2002 can include a textured surface that mimics the real anatomy of the patient. Additionally, the glenoid face 2002 can include a virtual boundary 2006 can be defined on a virtual surface 2004 of the glenoid face 2002. In certain implementations, the virtual boundary 2006 can be marked around the edges of a glenoid face 2002. In certain implementations, the virtual boundary 2006 can be visually represented in various ways, including, but not limited to, a solid line, a dotted line, or a shaded area. Additionally, the user interface 2000 presents a virtual representation of a surgical guide 2008. As shown, the surgical guide 2008 is positioned and aligned within the virtual boundary 2006 on the glenoid face 2002.

Similar to FIG. 20A, FIG. 20B depicts the user interface 2000 presenting a virtual representation of the glenoid and a surgical guide. However, FIG. 20B provides a posterior perspective. As depicted in FIG. 20B, the surgical guide 2008 includes a defined number of virtual prongs 2010 (e.g., three or any positive integer). In certain implementations, each virtual prong 2010 can be positioned on the virtual surface 2004 of the glenoid face 2002. As shown, each virtual prong 2010 establishes direct contact with the virtual surface 2004 of the glenoid face 2002, as depicted in greater detail in FIG. 20C.

Referring now to FIG. 20C, depicted is a close-up view of a surgical guide with virtual prongs resting on the virtual surface of a glenoid face. In certain implementations, when the virtual prongs 2010 rest on the glenoid surface 2004, this establishes contact points between the virtual prongs 2010 and the glenoid surface 2004. Based on the positions of the contact points, the virtual model of the surgical guide 2008 generates a glenoid plane 2012 that incorporates all three points. In certain implementations, the glenoid plane 2012 can provide the estimated orientation and shape of the glenoid surface 2004. In certain implementations, the glenoid plane 2012 can refer to a virtual plane generated based on the positions of the virtual prongs 2010 on the virtual surface 2004 of the glenoid face 2002. The glenoid plane 2012 can represent an approximation of the actual glenoid surface orientation or shape.

Upon establishing contact between the virtual prongs 2010 and the virtual surface 2004 of the glenoid face 2002 (as depicted in FIG. 20C), the user interface 2000 can display one or more angles to guide the drill trajectory. As illustrated, the version angle, represented by arc 2016, indicates the extent to which the axis of the virtual model of the surgical guide 2008 tilts backward relative to the glenoid plane 2012. Additionally, the inclination angle, represented by arc 2018, indicates the extent to which the axis of the virtual model of the surgical guide 2008 tilts downward relative to the glenoid plane 2012.

Referring now to FIG. 20D, depicted is a comparable close-up view to FIG. 20C, illustrating a different perspective view of an interaction with the virtual model of a surgical guide on the virtual surface of a glenoid face. As shown, a drill bit 2014 is pointed towards a drilling location in alignment with the version and inclination angles. In certain implementations, the user (e.g., a surgeon) can adjust the virtual model of the surgical guide 2008 based on visual feedback or displayed values to achieve the desired version and inclination angles. In certain implementations, the data processing system 1905 can adjust the virtual model of the surgical guide 2008 based on predefined settings or real-time calculations from patient data.

In certain implementations, the drill bit 2014 can be guided by the virtual model of a surgical guide 2008. In certain implementations, the data processing system 1905 (e.g., via a virtual guidance system) can provide visual or haptic feedback to guide the surgeon's hand while holding the drill bit 2014. To confirm alignment, the data processing system 1905 can provide visual feedback displaying the drill bit's position and trajectory relative to the glenoid plane 2012 and the version and inclination angles. Additionally, in certain implementations, the haptic feedback can provide physical resistance when the drill bit 2014 deviates from the desired path. In certain implementations, pressure sensors in the virtual prongs 2010 or the virtual model of the surgical guide 2008 can determine pressure on the virtual surface 2006 of the glenoid face 2002.

### C. Surgical System

The virtual model of the surgical guide described in Section B can be integrated into a surgical system. The present disclosure is directed towards incorporating the virtual model of the surgical guide into the surgical system to enhance surgical planning and execution. The integration of the virtual surgical guide model can facilitate a more precise approach to robotic-assisted surgeries. Additionally, the inclusion of the virtual model can enable the utilization of both the physical and virtual aspects of the surgical guide within the advanced technological environment of robotic surgery.

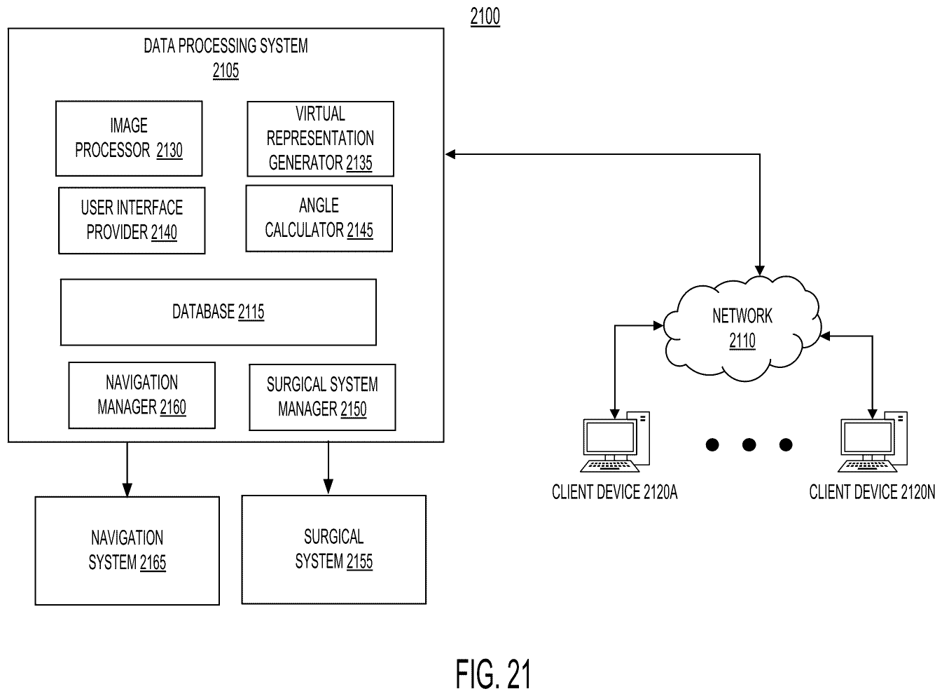

Referring now to FIG. 21, depicted is a block diagram of an example system 2100 comprising at least one data processing system 2105, a surgical system 2155, a network 2110, and one or more client devices 2120A-2120N (sometimes generally referred to as client device(s) 2120). Various components of the system 2100 shown in FIG. 21 may be similar to, and include any of the structure and functionality of, the system 1900 of FIG. 19. For example, the network 2110 shown in FIG. 21 is similar to the network 1910 in FIG. 19. Additionally, the data processing system 2105 may include some or all components from its counterpart in FIG. 19.

The data processing system 2105 can include a system manager 2150 (e.g., a robotic system manager), which can be a script, module, library, or function that manages and controls the surgical system 2155. In certain implementations, the surgical system manager 2150 can transfer the surgical instructions or virtual plan data tailored for operations (e.g., robotic operations) to the surgical system 2155. The transmission can occur through various methods, including, but not limited to, network communication or a direct hardware interface. In certain implementations, the surgical instructions or the virtual plan data can include a drilling location and a selected pathway for the surgical implant placement in the glenoid. Additionally, the virtual plan data can include the version and inclination angles of the virtual model of the surgical guide 2008 positioned relative to the virtual surface 2004 of the glenoid face 2002. For patient-specific surgical planning, the surgical system manager 2150 (or the surgical system 2155) can validate the surgical instructions or the virtual plan data against preoperative diagnostic data tailored to the patient's specific anatomical configurations.

In certain implementations, the surgical system manager 2150 can monitor or manage sensor data received from the surgical system 2155. The sensor data can correspond to a robotic surgical arm's position or orientation. In certain implementations, the surgical system manager 2150 can provide a feedback loop to monitor the surgical system's performance or deviations from planned movements. In certain implementations, the surgical system manager 2150 can predefine safety protocols to implement emergency shutdown procedures or limit movement zones. In certain implementations, the surgical system manager 2150 can connect with external systems, such as the network 2110 or the client device 2120, to allow for data exchange and user interaction.

The surgical system 2155 can include various components, including, but not limited to, a robotic surgical arm, a collection of surgical tools, a control system, and an array of sensors, among others. The robotic surgical arm can include multiple joints and motors, providing a wide range of motions and controlled manipulation within the surgical field. In certain implementations, the surgical system 2155 can include a variety of tools depending on the specific surgical needs, with each tool tailored for tasks such as cutting, grasping, or suturing.

Once the virtual plan data is received and/or validated, the surgical system 2155 can translate the virtual plan data into specific (e.g., real-time movement) commands. The surgical system 2155 can process the virtual plan data, extract key points, such as the desired path for drilling into the glenoid, and convert the key points into a series of joint angles and movement commands that can align with the structure or capabilities of the robotic surgical arm. In certain implementations, the surgical system 2155 can generate precise movements for the robotic surgical arm in response to identifying the position and orientation of the virtual model of the surgical guide 2008 relative to the virtual surface 2004 of the glenoid face 2012.

In certain implementations, the surgical system 2105 can receive instructions directly from a surgeon/user. The surgical system 2105, via the control system, can process or translate the instructions into specific movement commands, as described herein. In certain implementations, the surgical system 2105 can receive translated movement commands from the surgical system manager 2150. Once the movement commands are received, the surgical system 2105 can transmit the movement commands to the robotic surgical arm or the attached surgical tools. For example, in response to receiving the movement commands, the robotic surgical arm can position the surgical guide 100 (e.g., a physical surgical guide) or initiate a drill to perform the surgery at the designated drilling location.

In certain implementations, the surgical system 2105 can include sensors, which may include, but are not limited to, position sensors, force sensors, and visual feedback systems. In certain implementations, the position sensors can monitor the location of the robotic surgical arm relative to the patient's anatomy. In certain implementations, the force sensors can provide feedback on the amount of pressure being applied by the robotic surgical arm or the surgical tools. In certain implementations, the visual feedback systems, such as cameras and 3D imaging technologies, can provide a detailed view of the surgical site.

### D. Navigation System

The implementation of virtual surgical planning, as discussed in Section B, can be facilitated through a navigation system. The present disclosure is directed towards utilizing surgical navigation technology, which enables surgeons and clinicians to monitor the positions of surgical instruments. Additionally, the implementation can be extended to directing the operations of a surgical system. The present disclosure is applicable to and can be integrated with existing navigation systems, including those utilizing augmented reality (AR), virtual reality (VR), or mixed reality (MR) technologies. For example, the integration can enhance a surgeon's precision by overlaying detailed 3D anatomical structures directly onto their field of view during surgery, or it can simulate complex surgical procedures to provide feedback on instrument positioning or procedural steps.

Using the implementation illustrated in FIG. 21, the example system 2100 can include a navigation system 2165, managed by a navigation module 2160, within the data processing system 2105. The aforementioned configuration can provide integration of the navigation system 2165 within the scope of executing the virtual surgical plan. As illustrated in FIG. 21, the data processing system 2105 can include the navigation manager 2160, which can be a script, module, library, or function that manages or controls the navigation system 2165. The navigation manager 2160 can establish and maintain communication channels with the navigation system 2165 to exchange data for activities such as image acquisition, instrument tracking, or providing feedback, among others.

The navigation system 2165 can access and process information from the virtual surgical plan, which can include, but is not limited to, a virtual representation of the glenoid face 2002, complete with anatomical landmarks and precise implant placement details, the specific version and inclination angles of the virtual model of the surgical guide 2008, and any other relevant data related to the surgery. In certain implementations, the navigation system 2165 can receive intraoperative images of the glenoid of the patient. Intraoperative images of the glenoid can be captured using modalities such as fluoroscopy or CT scans.

In certain implementations, the navigation system 2165 can merge or overlay the virtual surgical plan (e.g., the virtual representation of the glenoid face 2002) with the intraoperative image to indicate the ideal (or target) location for a surgical implant. In certain implementations, the navigation system 2165 can identify the position of the drilling instrument (e.g., the drilling bit 2014) or the surgical guide 100 within the image. In certain implementations, the navigation system 2165 can identify the position of the surgical guide 100 (e.g., a physical surgical guide) using predefined marker patterns on the surgical guide 100. The predefined marker patterns can be embedded or engraved on one or more feet 118 of the surgical guide 100. In certain implementations, the predefined marker patterns can include, but are not limited to, geometric shapes (e.g., triangles, squares, circles), alphanumeric characters (e.g., letters, numbers), or color-coded markers. In certain implementations, the navigation system 2165 can align the virtual representation of the glenoid face 2002 with the patient's actual anatomy by matching identified landmarks (e.g., bony structures).

In certain implementations, when the navigation system 2165 identifies a match between the marker patterns on the surgical guide 100 and the corresponding coordinates in the virtual plan, the navigation system 2165 can provide feedback (e.g., via a visual indicator or an auditory alert), informing the user or surgeon of the alignment of the surgical guide 100 with the targeted anatomical site.

In certain implementations, the navigation system 2165 can determine the spatial relationship between the drilling instrument (e.g., the drill bit 2014) or the surgical guide 100 and the planned drilling location. In certain implementations, the navigation system 2165 can use preoperative data and the position of the drilling instrument to determine the spatial relationship. For example, by identifying the current position of the drilling instrument in relation to the virtual model of the patient's anatomy, the navigation system 2165 can determine how the surgical instrument's location corresponds to the planned drilling site. In certain implementations, the navigation system 2165 can identify the version angle and/or the inclination angle of the surgical guide 100. In certain implementations, the spatial parameters can include intersection points, defined as locations where the drilling instrument/surgical guide axis intersects the planned drilling path or target area. In certain implementations, the spatial parameters can include areas of overlap identified as regions where the drilling instrument and the planned drilling target area coincide. In certain implementations, the navigation system 2165 can present the spatial relationship in various forms, including, but not limited to, offset distances, angles, or directional vectors.

In certain implementations, the navigation system 2165 can generate dynamic indicators to identify the ideal drilling location on the glenoid. Additionally, in certain implementations, the navigation system 2165 can access a time-stamped sequence of the intraoperative images of the glenoid in response to identifying the movement of the drilling/surgical instrument. In certain implementations, the navigation system 2165 can present the indicators in several ways, including, but not limited to, projecting a light pattern to illuminate the exact spot where the drill bit 2014 should be placed, overlaying the location in the surgeon's augmented reality, virtual reality, or mixed reality headset, or displaying the indicator superimposed on the intraoperative image on a monitor. In certain implementations, the navigation system 2165 can transmit the visual indicators to the surgeon. In certain implementations, the navigation system 2165 can transmit the indicators to the surgical system 2155. In certain implementations, the surgical system 2155 can use the indicators provided by the navigation system 2165 to position the surgical guide 100 and/or the drilling bit 2014.

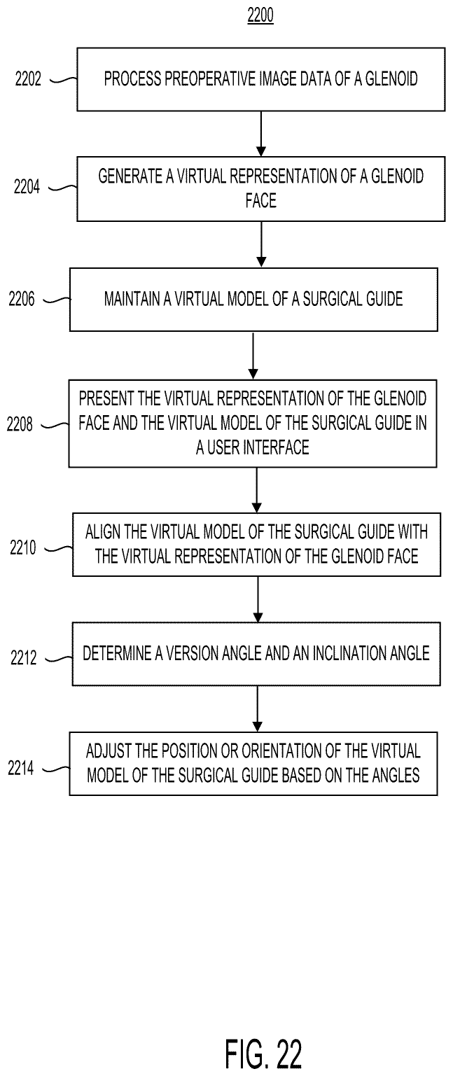

Referring now to FIG. 22 in the context of the components described in connection with FIGS. 19 and 21, depicted is an illustrative flow diagram of a method 2200 for providing a virtual surgical plan. The method 2200 can be performed by one or more processors depicted in FIGS. 19 and 21. A data processing system can process preoperative image data of a glenoid (STEP 2202). The data processing system can generate a virtual representation of the glenoid face (STEP 2204). The data processing system can maintain a virtual model of a surgical guide (STEP 2206). The data processing system can present the virtual representation of the glenoid face and the virtual model of the surgical guide in a user interface (STEP 2208). The data processing system can align the virtual model of the surgical guide with the virtual representation of the glenoid face (STEP 2210). The data processing system can determine a version angle and an inclination angle (STEP 2212). The data processing system can adjust the position or orientation of the virtual model of the surgical guide based on the angles (STEP 2214).

At 2202, a data processing system can process preoperative image data of a glenoid to identify defined anatomical landmarks. The defined anatomical landmarks can include a glenoid face of the glenoid. For example, the anatomical landmarks may include, but are not limited to, the anterior glenoid rim, posterior glenoid rim, and tubercle, among others.

At 2204, the data processing system can generate a virtual representation of the glenoid face based on defined anatomical landmarks. The virtual representation of the glenoid face can include a virtual boundary that can be adjusted/defined on a virtual surface of the glenoid face. The virtual representation can include a glenoid plane that can be positioned on the virtual surface of the glenoid face. The virtual plane can indicate the drilling trajectory.

The virtual boundary can be dynamically adjusted on the virtual surface of the glenoid face. The virtual boundary can represent the planned location and size of a surgical implant on the glenoid face. In certain implementations, the virtual boundary can be updated in real-time (manually or automatically) to show surgical instruments' (e.g., a surgical guide or a drilling bit) positions and other associated potential issues.

At 2206, the data processing system can maintain a virtual model of a surgical guide in a data structure. The data processing system can store a library of various surgical guide designs, where each design can be represented as a 3D model. Surgeons/Users can select or customize the appropriate surgical guide for a surgical procedure. The virtual model of the surgical guide can include a virtual base plane with a defined number of virtual prongs. The number of prongs can be three or any positive integer, depending on the implementation. Each virtual prong can be positioned on the virtual surface of the glenoid face within a virtual boundary. In certain implementations, the data structure can be shared with other systems involved in the surgical process, such as robotic arms or navigation systems, for coordinated planning and execution.

At step 2208, the data processing system can present the virtual representation of the glenoid face and the virtual model of the surgical guide in a user interface. The data processing system can display a view of the virtual representation of the glenoid face and the virtual model of the surgical guide for the user. In certain implementations, the data processing system can receive an interaction via the user interface, which allows for the movement of the position or orientation of the virtual model of the surgical guide. For example, in response to the interaction, the data processing system can modify the view of the virtual representation of the glenoid face and the virtual model of the surgical guide. In certain implementations, the modification can include changing the view to at least one of a coronal view, a sagittal view, or a transverse view. In certain implementations, the interaction can be received through various means such as an interactive element, a button, a voice command, a joystick, a gesture recognition system, or tactile/haptic feedback.

At step 2210, the data processing system can align the virtual model of the surgical guide with the virtual representation of the glenoid face. In certain implementations, the data processing system can align the virtual model of the surgical guide with a patient's pre-operative scans, such as CT images or 3D reconstructions, and can simulate placement scenarios on the patient's anatomy. The data processing system can position (e.g., based on a use request) the virtual model of the surgical guide within the virtual boundary. In certain implementations, the data processing system can automatically position the virtual prongs of the surgical guide at a first set of determined locations on the virtual surface of the glenoid face. In certain implementations, the data processing system can automatically reposition the virtual prongs of the surgical guide to a second set of determined locations on the virtual surface of the glenoid face in response to adjustments made to the position or orientation of the virtual model of the surgical guide.

At step 2212, the data processing system can determine a version angle and an inclination angle. For example, when the data processing system aligns the virtual model of the surgical guide with the virtual representation of the glenoid face, the data processing system can determine a version angle, which indicates a backward tilt or a forward tilt of an axis of the surgical guide relative to the glenoid plane. Additionally, the data processing system can determine an inclination angle, which indicates a downward tilt or an upward tilt of the axis of the surgical guide relative to the glenoid plane. In certain implementations, the data processing system can monitor the actual position of the surgical guide using imaging data. By comparing the actual position of the surgical guide to the planned angles, the data processing system can calculate deviations in version and inclination angles to identify any unintended tilting or misalignment of the surgical guide.

At step 2214, the data processing system can adjust the position or orientation of the virtual model of the surgical guide based on the angles (e.g., version and inclination angles). In certain implementations, the data processing system can automatically rotate the virtual model of the surgical guide around its axis (or a reference axis) to match the desired angles(s). In certain implementations, the data processing system can tilt the virtual guide model up or down based on the desired angle(s).

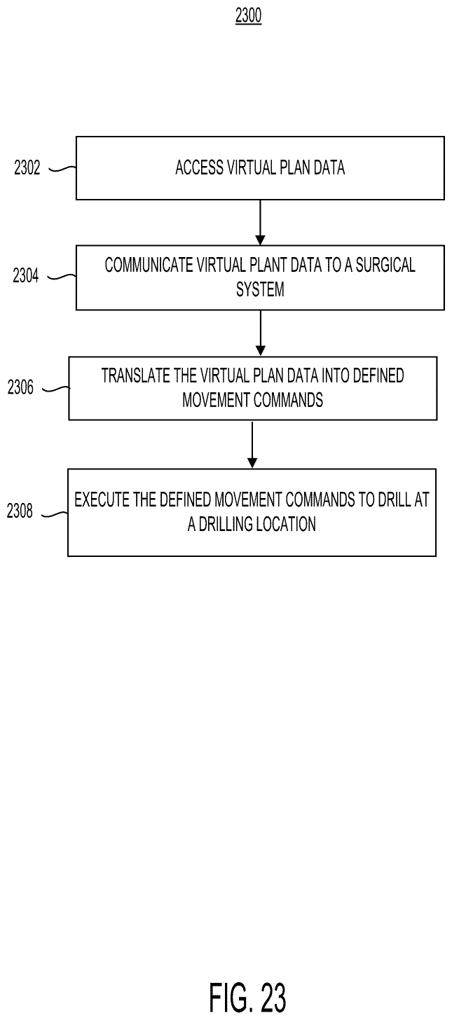

Referring now to FIG. 23 in the context of the components described in connection with FIGS. 19 and 21, depicted is an illustrative flow diagram of a method 2300 for executing a virtual surgical plan. The method 2300 can be performed by one or more processors depicted in FIGS. 19 and 21. A data processing system can access virtual plan data (STEP 2302). The data processing system can communicate the virtual plan data to a surgical system (STEP 2304). The surgical system can be configured to translate the virtual plan data into defined movement commands (STEP 2306). The surgical system can be configured to execute the defined movement commands to drill at a drilling location (STEP 2308).

At step 2302, the data processing system can access virtual plan data. In certain implementations, the virtual plan data can indicate a drilling location determined by defined anatomical landmarks, providing a pathway for drilling an opening to implant a surgical implant in the glenoid. The virtual plan data can indicate a version angle and an inclination angle for a virtual model of a surgical guide, positioned according to the drilling location and relative to a virtual representation of a glenoid face. The data processing system can validate the virtual plan data against preoperative data.

At step 2304, the data processing system can communicate the virtual plan data to a surgical system. The surgical system can include a surgical robotic arm that can execute the virtual surgical plan by manipulating surgical instruments according to the virtual plan data. In certain implementations, the data processing system can establish a network connection with the surgical system to provide the virtual plan data. In certain implementations, application programming interfaces (APIs) can facilitate communication between the data processing system and the surgical system. In certain implementations, the data processing system can transmit one or more elements of the virtual plan data, such as the surgical guide placement or the drilling location, to the surgical system.

At step 2306, the surgical system can translate the virtual plan data into defined movement commands. In certain implementations, the data processing system can perform the translation of the virtual plan data into defined movement commands for the surgical system. For example, the data processing system can identify the position or orientation of the virtual model of the surgical guide relative to the virtual representation of the glenoid face. In certain implementations, this may include a number of virtual prongs positioned on the virtual surface of the glenoid face. The surgical system can use the translated commands to perform the surgery accordingly.

At step 2308, the surgical system can execute the defined movement commands to drill at a drilling location. The surgical system can receive the translated movement commands and activate the motors in the robotic surgical arm. The robotic surgical arm can move the drilling instrument towards the target location on the glenoid face. Once the drilling instrument reaches the target location, the robotic system can execute the commands to control the drilling parameters, such as speed, pressure, and depth, based on the planned procedure or implant type.

In certain implementations, the data processing system can cause the surgical system to restrict the drilling instrument within the virtual boundary on the glenoid face. For example, during surgery, the surgical system can execute (e.g., via the robotic surgical arm) the translated commands while staying within the virtual boundary. One or more sensors track the position of the robotic surgical arm, and the surgical system or the data processing system can automatically adjust the movements of the robotic surgical arm to prevent accidental drilling outside the safe zone.

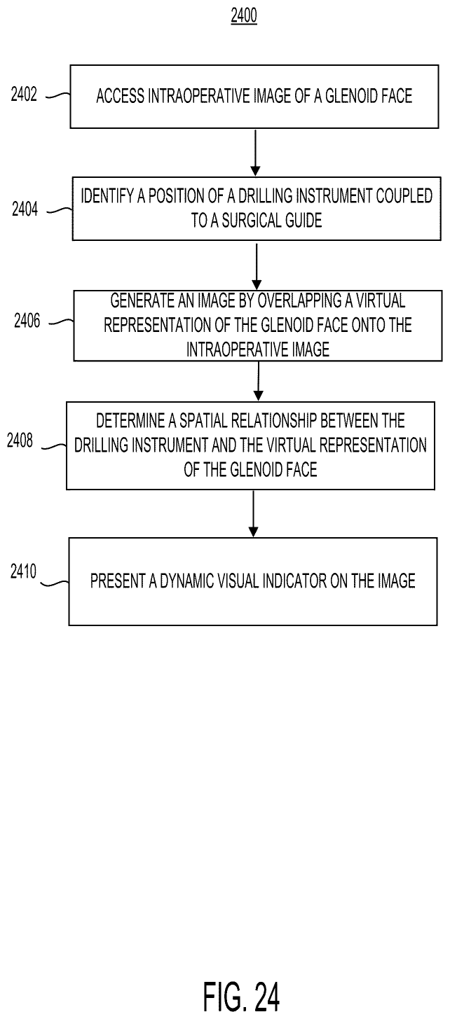

Referring now to FIG. 24 in the context of the components described in connection with FIGS. 19 and 21, depicted is an illustrative flow diagram of a method 2400 for navigating a virtual surgical plan. The method 2400 can be performed by one or more processors depicted in FIGS. 19 and 21. A data processing system can access an intraoperative image of a glenoid face (STEP 2402). The data processing system can identify a position of a drilling instrument coupled to a surgical guide (STEP 2404). The data processing system can generate an image by overlapping a virtual representation of the glenoid face onto the intraoperative image (STEP 2406). The data processing system can determine a spatial relationship between the drilling instrument and the virtual representation of the glenoid face (STEP 2408). The data processing system can present a dynamic visual indicator on the image (STEP 2410).

At step 2402, the data processing system can access an intraoperative image of a glenoid face. In certain implementations, the data processing system can be directly connected to an imaging system used during surgery, such as a fluoroscope or a navigation system, establishing a communication channel for receiving image data of the glenoid. In certain implementations, the data processing system can receive intraoperative images captured by the imaging system, which are uploaded to a cloud storage platform. The data processing system can access the platform through an internet connection to retrieve the image data.

At step 2404, the data processing system can identify the position of a drilling instrument coupled to a surgical guide within the intraoperative image. The data processing system can identify the position based on defined marker patterns of the surgical guide within the intraoperative image. In certain implementations, the defined marker patterns of the surgical guide can include geometric shapes (e.g., triangles, squares, or circles on the guide), alphanumeric characters (e.g., letters or numbers on the guide), or color-coded markers (e.g., incorporating different colored dots or patches on the guide). In response to determining the position of the markers relative to each other and their predefined locations on the surgical guide, the data processing system can determine the surgical instrument's position within the image and the surgical field.

At step 2406, the data processing system can generate an image by overlapping a virtual representation of the glenoid face onto the intraoperative image. The data processing system can identify anatomical landmarks in the virtual representation and the intraoperative image. The data processing system can align the landmarks. For example, before surgery, surgeons/users can use the virtual surgical planning software to generate a virtual model of the patient's glenoid face, which the data processing system can overlay onto the intraoperative image obtained during surgery. This overlay can be aligned based on identified anatomical landmarks to assist in visualizing the planned implant position or drilling trajectory in relation to the patient's actual anatomy. Additionally, as the surgery progresses, the data processing system can track/identify movements of the surgical guide/drilling instrument through sensors or marker patterns, as described herein.

At step 2408, the data processing system can determine a spatial relationship between the position of the drilling instrument and the virtual representation of the glenoid face using a version angle and an inclination angle of the surgical guide. The spatial relationship can indicate an offset, an angular deviation, or a depth difference between the drilling instrument and the desired drilling location on the glenoid face. In certain implementations, the offset can indicate the distance from the drilling instrument's tip (or the drill bit) to the target point on the virtual glenoid. In certain implementations, the angular deviation can indicate a discrepancy in orientation relative to the planned drilling path. In certain implementations, the depth difference can indicate a disparity between the drill bit and the specified drilling depth. If the drilling instrument's alignment deviates from the surgical plan, the data processing system can determine the adjustments for the drilling instrument/surgical guide to align the drilling instrument/surgical guide correctly with the target drilling point on the glenoid face.

At step 2410, the data processing system can present a dynamic visual indicator on the image to indicate the drilling location for positioning the drilling instrument. The data processing system can project a light pattern directly onto the glenoid face, where the light pattern visually indicates the drilling location or the exact spots where the drilling instrument is to be positioned for drilling. In certain implementations, the data processing system can overlay the drilling location onto a surgeon's view via an augmented reality, virtual reality, or mixed reality headset. Additionally, the data processing system can display the drilling location in relation to the virtual representation of the glenoid face on a monitor or screen. In certain implementations, the data processing system can generate a time-stamped sequence of intraoperative images of the glenoid face when it detects movement of the drilling instrument. The images can be reviewed by the surgeon or analyzed by the system to assess instrument trajectory, drilling progress, or other potential deviations from the planned path.

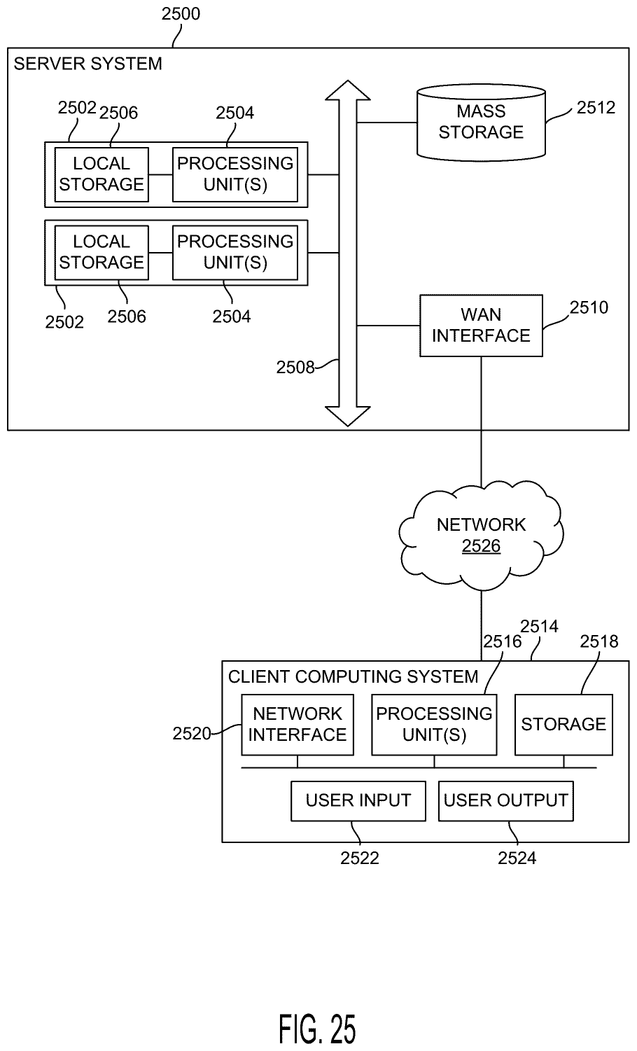

Various operations described herein can be implemented on computer systems. FIG. 25 shows a simplified block diagram of a representative server system 2500, client computer system 2514, and network 2526 usable to implement certain embodiments of the present disclosure. In various embodiments, server system 2500 or similar systems can implement services or servers described herein or portions thereof. Client computer system 2514 or similar systems can implement clients described herein. The system (e.g., the data processing system 1905, 2105) and others described herein can be similar to the server system 2500.

Server system 2500 can have a modular design that incorporates a number of modules 2502; while two modules 2502 are shown, any number can be provided. Each module 2502 can include processing unit(s) 2504 and local storage 2506.

Processing unit(s) 2504 can include a single processor, which can have one or more cores, or multiple processors. In some embodiments, processing unit(s) 2504 can include a general-purpose primary processor as well as one or more special-purpose co-processors such as graphics processors, digital signal processors, or the like. In some embodiments, some or all processing units 2504 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 2504 can execute instructions stored in local storage 2506. Any type of processors in any combination can be included in processing unit(s) 2504.

Local storage 2506 can include volatile storage media (e.g., DRAM, SRAM, SDRAM, or the like) and/or non-volatile storage media (e.g., magnetic or optical disk, flash memory, or the like). Storage media incorporated in local storage 2506 can be fixed, removable or upgradeable as desired. Local storage 2506 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 2504 need at runtime. The ROM can store static data and instructions that are needed by processing unit(s) 2504. The permanent storage device can be a non-volatile read-and-write memory device that can store instructions and data even when module 2502 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 2506 can store one or more software programs to be executed by processing unit(s) 2504, such as an operating system and/or programs implementing various server functions such as functions of the data processing system 1905 of FIG. 19 or any other system described herein, or any other server(s) associated with the data processing system 1905, or any other system described herein.

“Software” refers generally to sequences of instructions that, when executed by processing unit(s) 2504 cause server system 2500 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 2504. Software can be implemented as a single program or a collection of separate programs or program modules that interact as desired. From local storage 2506 (or non-local storage described below), processing unit(s) 2504 can retrieve program instructions to execute and data to process in order to execute various operations described above.

In some server systems 2500, multiple modules 2502 can be interconnected via a bus or other interconnect 2508, forming a local area network that supports communication between modules 2502 and other components of server system 2500. Interconnect 2508 can be implemented using various technologies including server racks, hubs, routers, etc.

A wide area network (WAN) interface 2500 can provide data communication capability between the local area network (interconnect 2508) and the network 2526, such as the Internet. Technologies can be used, including wired (e.g., Ethernet, IEEE 802.3 standards) and/or wireless technologies (e.g., Wi-Fi, IEEE 802.11 standards).

In some embodiments, local storage 2506 is intended to provide working memory for processing unit(s) 2504, providing fast access to programs and/or data to be processed while reducing traffic on interconnect 2508. Storage for larger quantities of data can be provided on the local area network by one or more mass storage subsystems 2512 that can be connected to interconnect 2508. Mass storage subsystem 2512 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 2512. In some embodiments, additional data storage resources may be accessible via WAN interface 2510 (potentially with increased latency).

Server system 2500 can operate in response to requests received via WAN interface 2510. For example, one of modules 2502 can implement a supervisory function and assign discrete tasks to other modules 2502 in response to received requests. Work allocation techniques can be used. As requests are processed, results can be returned to the requester via WAN interface 2510. Such operation can generally be automated. Further, in some embodiments, WAN interface 2510 can connect multiple server systems 2500 to each other, providing scalable systems capable of managing high volumes of activity. Techniques for managing server systems and server farms (collections of server systems that cooperate) can be used, including dynamic resource allocation and reallocation.

Server system 2500 can interact with various user-owned or user-operated devices via a wide-area network such as the Internet. An example of a user-operated device is shown in FIG. 4 as client computing system 2514. Client computing system 2504 can be implemented, for example, as a consumer device such as a smartphone, other mobile phone, tablet computer, wearable computing device (e.g., smart watch, eyeglasses), desktop computer, laptop computer, and so on.

For example, client computing system 2514 can communicate via WAN interface 2510. Client computing system 2514 can include computer components such as processing unit(s) 2516, storage device 2518, network interface 2520, user input device 2522, and user output device 2524. Client computing system 2514 can be a computing device implemented in a variety of form factors, such as a desktop computer, laptop computer, tablet computer, smartphone, other mobile computing device, wearable computing device, or the like.

Processing unit(s) 2516 and storage device 2518 can be similar to processing unit(s) 2504 and local storage 2506 described above. Suitable devices can be selected based on the demands to be placed on client computing system 2504; for example, client computing system 2514 can be implemented as a “thin” client with limited processing capability or as a high-powered computing device. Client computing system 2514 can be provisioned with program code executable by processing unit(s) 2516 to enable various interactions with server system 2500 of a message management service such as accessing messages, performing actions on messages, and other interactions described above. Some client computing systems 2504 can also interact with a messaging service independently of the message management service.

Network interface 2520 can provide a connection to the network 2526, such as a wide area network (e.g., the Internet) to which WAN interface 2510 of server system 2500 is also connected. In various embodiments, network interface 2520 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 2522 can include any device (or devices) via which a user can provide signals to client computing system 2504; client computing system 2514 can interpret the signals as indicative of particular user requests or information. In various embodiments, user input device 2522 can include any or all of a keyboard, touch pad, touch screen, mouse or other pointing device, scroll wheel, click wheel, dial, button, switch, keypad, microphone, and so on.

User output device 2524 can include any device via which client computing system 2514 can provide information to a user. For example, user output device 2524 can include a display to display images generated by or delivered to client computing system 2504. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 2524 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in the present disclosure can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 2504 and 2506 can provide various functionality for server system 2500 and client computing system 2504, including any of the functionality described herein as being performed by a server or client, or other functionality associated with message management services.

It will be appreciated that server system 2500 and client computing system 2514 are illustrative and that variations and modifications are possible. Computer systems used in connection with embodiments of the present disclosure can have other capabilities not specifically described here. Further, while server system 2500 and client computing system 2514 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

Implementations of the subject matter and the operations described in the present disclosure can be implemented in digital electronic circuitry, or in computer software embodied on a tangible medium, firmware, or hardware, including the structures disclosed in the present disclosure and their structural equivalents, or in combinations of one or more of them. Implementations of the subject matter described in the present disclosure can be implemented as one or more computer programs, e.g., one or more components of computer program instructions, encoded on computer storage medium for execution by, or to control the operation of, data processing apparatus. The program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of these. Moreover, while a computer storage medium is not a propagated signal, a computer storage medium can include a source or destination of computer program instructions encoded in an artificially-generated propagated signal. The computer storage medium can also be, or be included in, one or more separate physical components or media (e.g., multiple CDs, disks, or other storage devices).

The operations described in the present disclosure can be implemented as operations performed by a data processing apparatus on data stored on one or more computer-readable storage devices or received from other sources.

The terms “data processing apparatus”, “data processing system”, “client device”, “computing platform”, “computing device”, or “device” encompasses all kinds of apparatus, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of these. The apparatus and execution environment can realize various different computing model infrastructures, such as web services, distributed computing, and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in the present disclosure can be performed by one or more programmable processors executing one or more computer programs to perform actions by operating on input data and generating output. The processes and logic flows can also be performed by, and apparatuses can also be implemented as, special purpose logic circuitry, e.g., an FPGA or an ASIC.

Processors suitable for the execution of a computer program include, by way of example, both general and special purpose microprocessors, and any one or more processors of any kind of digital computer. Generally, a processor will receive instructions and data from a read-only memory or a random access memory or both. The elements of a computer include a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), for example. Devices suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a player, implementations of the subject matter described in the present disclosure can be implemented on a computer having a display device, e.g., a CRT (cathode ray tube), plasma, or LCD (liquid crystal display) monitor, for displaying information to the player and a keyboard and a pointing device, e.g., a mouse or a trackball, by which the player can provide input to the computer. Other kinds of devices can be used to provide for interaction with a player as well; for example, feedback provided to the player can include any form of sensory feedback, e.g., visual feedback, auditory feedback, or tactile feedback; and input from the player can be received in any form, including acoustic, speech, or tactile input. In addition, a computer can interact with a player by sending documents to and receiving documents from a device that is used by the player; for example, by sending web pages to a web browser on a player's client device in response to requests received from the web browser.

Implementations of the subject matter described in the present disclosure can be implemented in a computing system that includes a back-end component, e.g., as a data server, or that includes a middleware component, e.g., an application server, or that includes a front-end component, e.g., a client computer having a graphical user interface or a Web browser through which a player can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system such as the gaming system described herein can include clients and servers. For example, the gaming system can include one or more servers in one or more data centers or server farms. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving input from a player interacting with the client device). Data generated at the client device (e.g., a result of an interaction, computation, or any other event or computation) can be received from the client device at the server, and vice-versa.

While the present disclosure contains many specific implementation details, these should not be construed as limitations on the scope of any inventions or of what may be claimed, but rather as descriptions of features specific to particular implementations of the systems and methods described herein. Certain features that are described in the present disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. For example, the gaming system could be a single module, a logic device having one or more processing modules, one or more servers, or part of a search engine.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations, elements, or acts of the systems and methods herein referred to in the singular may also embrace implementations including a plurality of these elements; and any references in plural to any implementation, element, or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description, or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein may be embodied in other specific forms without departing from their characteristics thereof. The foregoing implementations are illustrative, rather than limiting, of the described systems and methods. The scope of the systems and methods described herein may thus be indicated by the appended claims, rather than the foregoing description, and changes that come within the meaning and range of equivalency of the claims are embraced therein.

As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. The devices, systems and methods described herein may be embodied in other specific forms without departing from the characteristics thereof. The foregoing implementations are illustrative rather than limiting of the described systems and methods. The scope of the systems and methods described herein is thus indicated by the appended claims, rather than the foregoing description, and changes that come within the meaning and range of equivalency of the claims are embraced therein.

1. A method for virtual surgical planning, comprising:
processing, by one or more processors, preoperative image data of a glenoid to identify defined anatomical landmarks, the defined anatomical landmarks including a glenoid face of the glenoid;
generating, by the one or more processors, a virtual representation of the glenoid face based on the defined anatomical landmarks, the visual representation including a dynamically adjustable virtual boundary defined on a virtual surface of the glenoid face;
maintaining, by the one or more processors, in a data structure, a virtual model of a surgical guide;
providing, by the one or more processors, for presentation, in a user interface, the virtual representation of the glenoid face and the virtual model of the surgical guide, the virtual model of the surgical guide configured to be aligned with the virtual representation of the glenoid face, wherein the virtual model of the surgical guide includes a virtual base plane defined by a number of virtual prongs, each virtual prong to be positioned on the virtual surface of the glenoid face within the virtual boundary such that the virtual base plane aligns with a glenoid plane defined by the virtual surface of the glenoid face;
in response to aligning the virtual model of the surgical guide with the virtual representation of the glenoid face, determining, by the one or more processors:
a version angle indicating at least one of a backward tilt or a forward tilt of an axis of the surgical guide relative to the glenoid plane; and
an inclination angle indicating at least one of a downward tilt or an upward tilt of the axis of the surgical guide relative to the glenoid plane; and
adjusting, by the one or more processors, a position or orientation of the virtual model of the surgical guide based on at least one of the version angle or the inclination angle.

2. The method of claim 1, further comprising:
presenting, by the one or more processors, for display, a view of the virtual representation of the glenoid face and the virtual model of the surgical guide;
receiving, by the one or more processors, an interaction, via the user interface, to move the position or orientation of the virtual model of the surgical guide; and
modifying, by the one or more processors, in response to receiving the interaction, the view of the virtual representation of the glenoid face and the virtual model of the surgical guide, wherein the view is at least one of a coronal view, a sagittal view, or a transverse view.

3. The method of claim 2, wherein the interaction is received via an interactive element, a button, a voice command, a joystick, a gesture recognition system, tactile feedback, or haptic feedback.

4. The method of claim 1, wherein the virtual base plane comprises at least three virtual prongs.

5. The method of claim 1, further comprising restricting, by the one or more processors, positioning of the virtual model of the surgical guide within the virtual boundary.

6. The method of claim 1, further comprising providing, by the one or more processors, feedback in response to determining that the virtual model of the surgical guide extends outside the virtual boundary, wherein the feedback includes at least one of an error message or a visual indication.

7. The method of claim 1, further comprising:
receiving, by the one or more processors, an input, via the user interface, to adjust the position or orientation of the virtual model of the surgical guide; and
in response to receiving the input, generating, by the one or more processors, an output identifying the version angle and the inclination angle.

8. The method of claim 1, further comprising automatically positioning, by the one or more processors, the virtual prongs of the virtual model of the surgical guide at a first set of determined prong locations on the virtual surface of the glenoid face.

9. The method of claim 8, further comprising automatically repositioning, by the one or more processors, the virtual prongs of the virtual model of the surgical guide to a second set of determined prong locations on the virtual surface of the glenoid face in response to adjusting the position or orientation of the virtual model of the surgical guide.

10. The method of claim 1, wherein the surgical guide comprises:
a base including at least three feet defining a base plane;
a drill guide coupled to the base, the drill guide including a drill guide body defining a drill bore configured to receive a drill bit and extending through the drill guide, the drill guide being configured to translate relative to the base to adjust a drill guide angle defined by the drill bore and the base, the drill guide further being configured to transition between a locked state and an unlocked state, wherein the drill guide angle is fixed in the locked state and the drill guide angle is adjustable in the unlocked state, the drill guide angle being defined by the drill bore and the base plane;
a first track coupled to the base and defining a first track opening; and
a second track coupled to the base and defining a second track opening, wherein the drill guide is configured to translate relative to the base within the first track opening and the second track opening.

11. The method of claim 1, further comprising indicating, by the one or more processors, varying anatomical densities on the virtual representation of the glenoid face with color-coded regions, the varying anatomical densities determined from the preoperative image data of the glenoid.

12. The method of claim 1, further comprising determining, by the one or more processors, the version angle and the inclination angle based on biomechanical data indicating a patient-specific glenoid anatomy.

13. The method of claim 1, further comprising determining, by the one or more processors, a spatial relationship between the virtual model of the surgical guide and the virtual representation of the glenoid face, wherein the spatial relationship includes one or more spatial parameters indicating at least one of a distance between the virtual base plane of the surgical guide and the glenoid plane, orientation angles, intersection points, or areas of overlap.

14. A system, comprising:
one or more processors coupled with memory and configured to:
process preoperative image data of a glenoid to identify defined anatomical landmarks, the defined anatomical landmarks including a glenoid face of the glenoid;
generate a virtual representation of the glenoid face based on the defined anatomical landmarks, the visual representation including a dynamically adjustable virtual boundary defined on a virtual surface of the glenoid face;
maintain, in a data structure, a virtual model of a surgical guide;
provide for presentation, in a user interface, the virtual representation of the glenoid face and the virtual model of the surgical guide, the virtual model of the surgical guide configured to be aligned with the virtual representation of the glenoid face, wherein the virtual model of the surgical guide includes a virtual base plane defined by a number of virtual prongs, each virtual prong to be positioned on the virtual surface of the glenoid face within the virtual boundary such that the virtual base plane aligns with a glenoid plane defined by the virtual surface of the glenoid face;
determine, in response to aligning the virtual model of the surgical guide with the virtual representation of the glenoid face:
a version angle indicating at least one of a backward tilt or a forward tilt of an axis of the surgical guide relative to the glenoid plane; and
an inclination angle indicating at least one of a downward tilt or an upward tilt of the axis of the surgical guide relative to the glenoid plane; and
adjust a position or orientation of the virtual model of the surgical guide based on at least one of the version angle or the inclination angle.

15. The system of claim 14, wherein the one or more processors are configured to:
present, for display, a view of the virtual representation of the glenoid face and the virtual model of the surgical guide;
receive an interaction, via the user interface, to move the position or orientation of the virtual model of the surgical guide; and
modify, in response to receiving the interaction, the view of the virtual representation of the glenoid face and the virtual model of the surgical guide, wherein the view is at least one of a coronal view, a sagittal view, or a transverse view.

16. The system of claim 15, wherein the interaction is received via an interactive element, a button, a voice command, a joystick, a gesture recognition system, tactile feedback, or haptic feedback.

17. The system of claim 14, wherein the virtual base plane comprises at least three virtual prongs.

18. The system of claim 14, wherein the one or more processors are configured to restrict positioning of the virtual model of the surgical guide within the virtual boundary.

19. The system of claim 14, wherein the one or more processors are configured to provide feedback in response to determining that the virtual model of the surgical guide extends outside the virtual boundary, wherein the feedback includes at least one of an error message or a visual indication.

20. The system of claim 14, wherein the one or more processors are configured to:
receive an input, via the user interface, to adjust the position or orientation of the virtual model of the surgical guide; and
in response to receiving the input, generate an output identifying the version angle and the inclination angle.

21-51. (canceled)
